(19) United States
(12) Patent Application Publication
(10) Pub. No.: US 2023/0399375 A1
(43) Pub. Date: Dec. 14, 2023

(54) METHODS AND COMPOSITIONS FOR TREATING AUTOIMMUNE DISEASE

(71) Applicant: Kyverna Therapeutics, Inc., Emeryville, CA (US)

(72) Inventors: Jeffrey Greve, Berkeley, CA (US); Lih-Yun Hsu, San Francisco, CA (US)

(21) Appl. No.: 18/300,919

(22) Filed: Apr. 14, 2023

Related U.S. Application Data
(60) Provisional application No. 63/331,397, filed on Apr. 15, 2022.

(51) Int. Cl. C07K 14/705; C07K 16/28; C12N 15/63

(52) U.S. Cl. CPC C07K 14/70553; C07K 16/2896; C07K 16/2845; C12N 15/63; C07K 2319/03; C07K 2319/715; C07K 2317/55; C07K 2317/622; C07K 2317/24

(57) ABSTRACT

This disclosure provides for chimeric receptor polypeptides where upon binding of a first antigen to the antigen binding domain triggers a proteolytic cleavage and upregulation of a chimeric antigen receptor, wherein the chimeric antigen receptor binds to a second antigen. The first and second antigens are present on a population of B cells known as Autoimmune- or Age-related B cells or CD11c+T-bet+ B cells. The present disclosure includes methods and compositions for reducing or eliminating ABCs through the binding of the chimeric antigen receptor, and thus controlling or eliminating autoimmune disease.

### CROSS-REFERENCE TO RELATED APPLICATIONS

This application claims priority to U.S. Provisional Patent Application No. 63/331,397, filed on Apr. 15, 2022. The contents of that application are incorporated herein by reference in its entirety.

### SEQUENCE LISTING

This application contains a Sequence Listing that has been submitted electronically as an XML file named 47902-0006WO1_SL_ST26.xml. The XML file, created on Apr. 11, 2023, is 265,637 bytes in size. The material in the XML file is hereby incorporated by reference in its entirety.

### TECHNICAL FIELD

This document relates to methods and compositions for treating disease, such as autoimmune disease, using cells expressing a chimeric receptor polypeptide and a chimeric antigen receptor polypeptide.

### BACKGROUND

Autoimmune disease is very common in the United States, with more than 20 million people suffering from at least one of 81 known autoimmune diseases. While autoimmune diseases can be treated with immunosuppressive drugs, there is currently no cure. B cells are known to be involved in different aspects of autoimmune diseases and are thought to contribute in a number of ways, including the secretion of autoantibodies, processing and presentation of autoantigen to T cells, as well as producing inflammatory cytokines. Thus, B cells are a target for the treatment of autoimmune diseases.

Within the last decade, a novel type of B cell associated with older animals was identified and termed Age Associated B-Cells, or ABCs (Rubtsov et al., Blood 2011, 118(5):1305-1315). These cells also have other names, including Double Negative B cells, Atypical Memory B-cells, and Tissue-like Memory B-Cells. ABCs have since been shown to express the cell surface receptor CD11c and the T-Box transcription factor, T-bet, and therefore they are now referred to as CD11c+T-bet +B cells. Unlike other B cells, ABCs express high levels of CD11c, a receptor typically expressed in myeloid cells, and the T-bet transcription factor that is known for its role as a master transcription factor regulating commitment of T cells to the T helper 1 (Th1) cell lineage commitment. T-bet is a key player in establishing and maintaining the phenotype. (Rubtsov et al., supra). In addition, high levels of T-bet expression are observed upon activation of the B cell antigen receptor (BCR) or IFN-γ receptor (Rubtsova et al., Cell Immunol 2015, 294(2):80-83).

### SUMMARY

This disclosure relates to methods and compositions for treating a patient with symptoms of an autoimmune disorder.

In some embodiments, provided herein are methods of modulating signaling in a cell. The cell may be administered to a patient in order to alleviate the symptoms of an autoimmune disorder. In some cases, the cell is transformed with a nucleic acid sequence encoding a chimeric receptor polypeptide, wherein the chimeric receptor polypeptide comprises an extracellular domain, a transmembrane domain, and an intracellular domain, wherein the extracellular domain comprises a first antigen binding domain capable of binding to a first antigen on a CD11c⁺Tbet⁺ B cell, and wherein the intracellular domain comprises a transcriptional control unit and a proteolytic site, a nucleic acid sequence encoding a chimeric antigen receptor polypeptide, wherein the chimeric antigen receptor comprises a second antigen binding domain capable of binding to a second antigen present on the CD11c⁺T-bet⁺ Bet cell, wherein the nucleic acid sequence encoding the chimeric antigen receptor polypeptide is operably linked to a transcriptional control element to which the transcriptional control unit can bind. In some cases, the cell is contacted with a CD11c+T-bet+ B cell expressing the first antigen on its surface, wherein the contacting induces cleavage at the proteolytic site, thereby releasing the intracellular domain. In some cases, releasing the intracellular domain result in the transcriptional control unit's activation of the transcriptional control element. The transcriptional control element is operably linked to the nucleic acid sequence encoding the chimeric antigen receptor polypeptide, which results in expression of the chimeric antigen receptor polypeptide.

In some embodiments, the chimeric receptor polypeptide is a chimeric NOTCH receptor polypeptide. In some embodiments, the chimeric NOTCH receptor is a SYNNOTCH® receptor.

In some embodiments, the first antigen binding domain is an antibody or antigen binding fragment. In some embodiments, the antibody binding fragment is selected from the group consisting of a Fab, a F(ab′)₂ fragment, a scFv, a scab, a dAb, a single domain heavy chain antibody, and a single domain light chain antibody.

In some embodiments, the first antigen is a B cell receptor. In some embodiments, the B cell receptor the B cell receptor is selected from the group consisting of CD19, CD20, and CD45R.

In some embodiments, the first antigen binding domain binds CD19. In some embodiments, the first antigen binding domain comprises an scFv comprising a sequence at least 90% identical to one of SEQ ID NOs: 1-10. In some embodiments, the first antigen binding domain comprises one of the following: (a) a heavy chain variable domain comprising SEQ ID NO: 39 and a light chain variable domain comprising SEQ ID NO: 77; (b) a heavy chain variable domain comprising SEQ ID NO: 40 and a light chain variable domain comprising SEQ ID NO: 78; (c) a heavy chain variable domain comprising SEQ ID NO: 41 and a light chain variable domain comprising SEQ ID NO: 79; (d) a heavy chain variable domain comprising SEQ ID NO: 42 and a light chain variable domain comprising SEQ ID NO: 80; (e) a heavy chain variable domain comprising SEQ ID NO: 43 and a light chain variable domain comprising SEQ ID NO: 81; (f) a heavy chain variable domain comprising SEQ ID NO: 44 and a light chain variable domain comprising SEQ ID NO: 82; (g) a heavy chain variable domain comprising SEQ ID NO: 45 and a light chain variable domain comprising SEQ ID NO: 83; (h) a heavy chain variable domain comprising SEQ ID NO: 46 and a light chain variable domain comprising SEQ ID NO: 84; (i) a heavy chain variable domain comprising SEQ ID NO: 47 and a light chain variable domain comprising SEQ ID NO: 85; or (j) a heavy chain variable domain comprising SEQ ID NO: 48 and a light chain variable domain comprising SEQ ID NO: 86.

In some embodiments, the second antigen is a receptor present on CD11c⁺T-bet B⁺ cells. In some embodiments, the second antigen binding domain is an antibody or antigen binding fragment. In some embodiments, the antibody fragment is selected from the group consisting of a Fab, a F(ab′)₂ fragment, a scFv, a scab, a dAb, a single domain heavy chain antibody and a single domain light chain antibody. In some embodiments, the second antigen is a receptor present on CD11c⁺T-bet B⁺ cells. In some embodiments, the second antigen is CD11c.

In some embodiments, the first antigen binding domain comprises an scFv comprising a sequence at least 90% identical to SEQ ID NO: 37 or SEQ ID NO: 38. In some embodiments, the first antigen binding domain comprises either (a) a heavy chain variable domain comprising SEQ ID NO: 75 and a light chain variable domain comprising SEQ ID NO: 113; or (b) a heavy chain variable domain comprising SEQ ID NO: 76 and a light chain variable domain comprising SEQ ID NO: 114.

In some embodiments, the proteolytic site is cleavable by a member of the ADAM family of proteases. In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence.

In some cases, the transformed cell is genetically modified with a nucleic acid comprising a nucleotide sequence encoding a chimeric antigen receptor (CAR), and wherein the intracellular domain of the mutated chimeric NOTCH polypeptide is a transcriptional activator or repressor. In some cases, the nucleic acid sequence encoding the CAR is operably linked to a transcriptional control element that is activated by the intracellular domain of the mutated chimeric NOTCH polypeptide. In some embodiments, the intracellular domain comprises a transcriptional control unit that comprises a transcriptional activator.

In some aspects, the first antigen binding domain is targeted to a first epitope and the second antigen binding domain is targeted to a second epitope. In some embodiments, the first epitope and the second epitope are on the same target, either on the same cell or same type of cells. In some embodiments, the first epitope and the second epitope are on different targets, either on the same cell or same type of cell. In some embodiments, the first antigen binding domain and the second antigen binding domain bind the same antigen and the same epitope.

Also provided herein are nucleic acids sequences that encode any of the chimeric antigen receptor polypeptides described herein. Also provided herein are vectors that include any of the nucleic acids encoding any of the chimeric antigen receptor polypeptides described herein.

Any of the vectors described herein can be an expression vector. For example, an expression vector can include a promoter sequence operably linked to the sequence encoding the chimeric antigen receptor polypeptides. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the immuno-activatable cells as described herein. Any appropriate promoter (e.g., EF1 alpha) can be operably linked to any of the nucleic acid sequences described herein. As used herein, the term “operably linked” is well known in the art and refers to genetic components that are combined such that they carry out their normal functions. For example, a gene is operably linked to a promoter when its transcription is under the control of the promoter. In another example, a nucleic acid sequence can be operable linked to another nucleic acid sequence by a self-cleaving 2A polypeptide. In such cases, the self-cleaving 2A polypeptide allows the second nucleic acid to be under the control of the promoter operably linked to the first nucleic acid sequence and allows the second nucleic acid to be in frame with the first nucleic acid.

In some cases, an exemplary nucleic acid sequence used to make an immuno-activatable cell as described herein can include a promoter operably linked to nucleic acid sequences encoding a CAR comprising an antigen binding domain capable of binding to antigen on a CD11c⁺ Tbet⁺ B cell, a CD8α transmembrane domain comprising a CD8α stalk and a CD8α hinge region, and a cytoplasmic signaling domain. In some cases, an exemplary nucleic acid sequence used make an immuno-activatable cell as described herein can include a promoter operably linked to nucleic acid sequences encoding a CAR comprising an antigen binding domain capable of binding to antigen on a CD11c⁺Tbet⁺ B cell, a CD8α transmembrane domain comprising a CD8α stalk and a CD8α hinge region, and a co-stimulatory domain. In some cases, an exemplary nucleic acid sequence used to make an immuno-activatable cell as described herein can include a promoter operably linked to nucleic acid sequences encoding a CAR comprising an antigen binding domain capable of binding to antigen on a CD11c⁺Tbet⁺ B cell, a CD8α transmembrane domain comprising a CD8α stalk and a CD8α hinge region, and a cytoplasmic signaling domain with a self-cleaving 2A sequence (e.g., a P2A, a T2A, a E2A or a F2A) (FIG. 2). For example, a nucleic acid sequence used to make an immuno-activatable cell can include sequences encoding a CAR comprising an antigen binding domain capable of binding to antigen on a CD11c⁺Tbet⁺ B cell, a CD8α transmembrane domain comprising a CD8α stalk and a CD8α hinge region, a cytoplasmic signaling domain, operably linked to the CAR (e.g., in frame) with a self-cleaving 2A sequence (e.g., a P2A, a T2A, a E2A or a F2A).

In some embodiments the T2A cleavage sequence (GSGEGRGSLLTCGDVEENPGP (SEQ ID NO: 280)), a P2A cleavage sequence (GSGATNFSLLKQAGDVEENPGP (SEQ ID

NO: 281)), a E2A cleavage sequence (GSGQCTNYALLKLAGDVESNPGP (SEQ ID NO: 282)) or a F2A cleavage sequence GSGVKQTLNFDLLKLAGDVESNPGP (SEQ ID NO: 283)).

Hinge domains may be derived from CD8, CD8α, CD4, CD28, 4-1BB, or IgG (in particular, the hinge domain of an IgG, for example from IgG1, IgG2, IgG3, or IgG4), and from an antibody heavy-chain constant region. Alternatively, the hinge domain may be a synthetic sequence.

In some cases, the nucleic acid sequences are in separate vectors. Alternatively, the nucleic acid sequences are included in the same vector. In some embodiments, provided herein are methods of treating a mammal having a disease, the method comprising administering to the mammal a cell transformed with the nucleic acids encoding the chimeric receptor polypeptide and the chimeric antigen receptors. In some embodiments, the cells used for treating the disease are transformed with the vectors comprising the nucleic acids encoding the chimeric receptor polypeptide and the chimeric antigen receptors. In some embodiments, the disease is an autoimmune disorder. For example, the autoimmune disorder may be selected from a group consisting of lupus, rheumatoid arthritis, multiple sclerosis, insulin dependent diabetes mellitus, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, and polyarteritis nodosa.

In some embodiments, provided herein are compositions for a chimeric receptor polypeptide comprising an extracellular domain, a transmembrane domain, and an intracellular domain, wherein the extracellular domain comprises an antigen binding domain that binds antigens present on CD11c⁺Tbet⁺B cells, wherein the intracellular domain comprises a transcriptional control unit and a proteolytic site, and wherein the transcriptional control unit comprises a domain capable of activating a transcriptional control element. In some embodiments, the chimeric receptor polypeptide is a chimeric NOTCH receptor polypeptide.

In some embodiments, the chimeric NOTCH receptor is a SYNNOTCH® receptor. In some aspects, the antigen binding domain is an antibody or antigen binding fragment. In some embodiments, the antibody or antigen binding fragment binds to a B cell receptor selected from the group consisting of CD19, CD20, and CD45R. In some embodiments, the antigen binding domain binds CD19. In some embodiments, the antigen binding domain comprises an scFv comprising a sequence at least 90% identical to one of SEQ ID NOs: 1-10. In some embodiments, the antigen binding domain comprises one of the following: (a) a heavy chain variable domain comprising SEQ ID NO: 39 and a light chain variable domain comprising SEQ ID NO: 77; (b) a heavy chain variable domain comprising SEQ ID NO: 40 and a light chain variable domain comprising SEQ ID NO: 78; (c) a heavy chain variable domain comprising SEQ ID NO: 41 and a light chain variable domain comprising SEQ ID NO: 79; (d) a heavy chain variable domain comprising SEQ ID NO: 42 and a light chain variable domain comprising SEQ ID NO: 80; (e) a heavy chain variable domain comprising SEQ ID NO: 43 and a light chain variable domain comprising SEQ ID NO: 81; (f) a heavy chain variable domain comprising SEQ ID NO: 44 and a light chain variable domain comprising SEQ ID NO: 82; (g) a heavy chain variable domain comprising SEQ ID NO: 45 and a light chain variable domain comprising SEQ ID NO: 83; (h) a heavy chain variable domain comprising SEQ ID NO: 46 and a light chain variable domain comprising SEQ ID NO: 84; (i) a heavy chain variable domain comprising SEQ ID NO: 47 and a light chain variable domain comprising SEQ ID NO: 85; or (j) a heavy chain variable domain comprising SEQ ID NO: 48 and a light chain variable domain comprising SEQ ID NO: 86.

The B cell receptor may be a receptor present on CD11c⁺T-bet B+cells.

In some cases, provided herein is the composition of a chimeric antigen receptor polypeptide comprising (i) a single-chain variable fragment (scFv) having binding specificity for a CD11c⁺T-Bet+ B cell antigen, (ii) a transmembrane domain, (iii) at least one co-stimulatory domain, and (iv) an activating domain. In some embodiments, scFV domain of the chimeric antigen receptor has binding specificity for CD11c.

In some embodiments, the scFv fragment comprises a sequence at least 90% identical to SEQ ID NO: 37 or SEQ ID NO: 38. In some embodiments, the scFv fragment comprises either: (a) a heavy chain variable domain comprising SEQ ID NO: 75 and a light chain variable domain comprising SEQ ID NO: 113; or (b) a heavy chain variable domain comprising SEQ ID NO: 76 and a light chain variable domain comprising SEQ ID NO: 114.

An isolated nucleic acid encoding any of the chimeric antigen receptor polypeptides described herein. In some embodiments, provided herein are pharmaceutical compositions comprising the chimeric receptor polypeptides and chimeric antigen receptors polypeptides as disclosed herein. In some embodiments, provided herein are isolated nucleic acid encoding the chimeric receptor polypeptides and chimeric antigen receptor polypeptides as disclosed herein. In some embodiments, provided herein are vectors comprising the chimeric receptor polypeptides and chimeric antigen receptor polypeptides as disclosed herein. In some embodiments, provided herein are the cells comprising the nucleic acids and vectors as described herein. In some embodiments, provided herein are methods for producing the chimeric receptor polypeptides and chimeric antigen receptors polypeptides from the nucleic acids, vectors and cells as described herein.

In some embodiments, the cell comprising the nucleic acid and vectors as described herein is an immune cell selected from the group consisting of a T cell, a B cell, a monocyte, a natural killer cell, a dendritic cell, a macrophage, a regulatory T cell, a helper T cell, and a cytotoxic T cell.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagram showing combinatorial recognition of CD11c+T-bet+B cells with a cell expressing the chimeric receptor polypeptides and chimeric antigen receptor as described herein. The CD11c+T-bet+B cells express CD19 and CD11c on the surface. The CAR-T cell, which expressed the chimeric receptor polypeptide binding to CD19, also express the chimeric antigen receptor, which here includes an antigen binding fragment capable of binding to CD11c. The entire process is predicated upon the binding of CD19 to the chimeric receptor polypeptide, cleavage of the intracellular domain, activation of expression of the chimeric antigen receptor, wherein the transcriptional control unit of the intracellular domain drove expression through control over the transcriptional control element. Additional candidate Pan-B cell receptors include CD19, CD20 and CD45R. An example of antigen bound by the antigen binding fragment is CD11c.

FIG. 2 is a diagram showing the steps of NOTCH receptor activation. The key structural features of Notch receptors are illustrated at left. S1 is the furin cleavage site. The center image illustrates interaction with a Notch ligand and the “pulling” force due to Notch ligand engagement on an adjacent cell, which unmasks the S2 ADAM cleavage site. S2 cleavage allows S3 gamma-secretase cleavage to occur. The right image illustrates release of the Notch ECD and ICD. Release of the ICD enables activation of Notch gene expression program.

FIGS. 3A-C shows the domain organization of SYNNOTCH® receptor compared to NOTCH. SS: Signal Sequence; NECD: NOTCH Extracellular Domain; NRR: Negative Regulatory Region; TM: Transmembrane domain; tTA-VP64: tetR-VP64 transcriptional activator.

### DETAILED DESCRIPTION

This document provides methods and compositions for treating a disease in a patient, including autoimmune disorders or cancer, by administering to the patient a cell transformed with a chimeric receptor polypeptide and a chimeric antigen receptor. In some embodiments, contacting the chimeric receptor polypeptide with a first antigen results in a cascade of signaling, resulting in the activation of expression of a chimeric antigen receptor. In some embodiments, the chimeric antigen receptor binds to a second antigen present either on the same cell or a different cell as the first antigen. In some embodiments, the antigen binding to the antigen binding fragment of the chimeric antigen receptor results in elimination of the cell expressing the second antigen.

The term “chimeric antigen receptor” or “CAR” as used herein generally refers to chimeric polypeptides containing, from amino to carboxy terminus, a light chain variable region and a heavy chain variable region, a transmembrane domain, a costimulatory signaling region, 4-1bb, OX40, or CD28, and an activating domain such as CD3 zeta, or fragments or functional mutants of these. See, for example, Geyer, Cytotherapy 2016, 18(11):1393-1409, and U.S. Pat. Nos. 7,741,465; 7,446,190; 9,605,049; 8,399,645; and 9,856,322, each of which is incorporated herein by reference in their entireties. It will be understood that there are other costimulatory signaling regions that can be used.

In some embodiments, the scFv comprises a light chain variable domain comprising a sequence that is at least 90% identical (e.g., at least 91%, 92%, 93%, 94%, 95%, 96%, 97%, 98%, 99%, or 100% identical) to one of SEQ ID NOs: 77-114. In some embodiments, the scFv comprises a heavy chain variable domain comprising a sequence that is at least 90% identical (e.g., at least 91%, 92%, 93%, 94%, 95%, 96%, 97%, 98%, 99%, or 100% identical) to one of SEQ ID NOs: 39-76.

As used herein, the term “activation” refers to induction of a signal on an immune cell (e.g., a B cell or T cell) that can result in initiation of the immune response (e.g., T cell activation). In some cases, upon binding of an antigen (e.g., CD19 or CD11c) to a T cell receptor (TCR) or an exogenous chimeric antigen receptor (CAR), the immune cell can undergo changes in protein expression that result in the activation of the immune response. In some cases, a TCR or CAR includes a cytoplasmic signaling sequence that can drive T cell activation. For example, upon binding of the antigen, a chimeric antigen receptor comprising an intracellular domain that includes a cytoplasmic signaling sequence (e.g., an immunoreceptor tyrosine-based inhibition motifs (ITAM)) that can be phosphorylated. A phosphorylated ITAM results in the induction of a T cell activation pathway that ultimately results in a T cell immune response. Examples of ITAMs include, without limitation, CD3 gamma, CD3 delta, CD3 epsilon, TCR zeta, FcR gamma, FcR beta, CD5, CD22, CD79a, and CD66d.

Ligand binding, or antigen binding domain in the context of chimeric NOTCH receptors refers to the substitution of a natural NOTCH ligand binding domain, e.g., EGF repeat sequences, with a non-natural ligand binding domain. Examples of the latter include antibodies, such as an scFv that binds its cognate antigen. There are a large number of other examples in which a ligand binding domain that binds to its cognate ligand that can be used to activate NOTCH receptor activity. These include, without limitation, growth factor receptors that bind their corresponding growth factors, etc. Another example is Affibodies. See, U.S. Pat. Nos. 6,740,734 and 6,602,977, and WO 00/63243, each of which is incorporated herein by reference in their entireties.

In some embodiments, a CAR can include a transmembrane domain. The transmembrane domain may be derived from a natural polypeptide, or may be artificially designed. If the transmembrane domain is derived from a natural polypeptide it can be obtained from a membrane-binding or transmembrane protein. For example, useable transmembrane domains can be from a T cell receptor alpha or beta chain, a CD3 zeta chain, CD28, CD3-epsilon, or numerous others known in the art. See, U.S. Pat. Nos. 9,670,281 and 9,834,608, both of which are incorporated by reference in their entireties. In some embodiments, the transmembrane domain is derived from CD28 or CD8. In some embodiments where the chimeric antigen receptor polypeptide includes a CD8 alpha transmembrane domain, the CD8 alpha transmembrane domain has an amino acid sequence is at least 80% (e.g., at least 85%, 90%, 95%, 99% and 100%) identical to NCBI Reference No: NP_001759 or a fragment thereof. In some embodiments where the chimeric antigen receptor polypeptide includes a CD28 transmembrane domain, the CD28 transmembrane domain has an amino acid sequence that is at least 80% (e.g., at least 85%, 90%, 95%, 99% and 100%) identical to SEQ ID NO: 277.

In some embodiments where the chimeric antigen receptor polypeptide includes a CD8 alpha transmembrane domain, the CD8 alpha transmembrane domain has an amino acid sequence that is at least 80% (e.g., at least 85%, 90%, 95%, 99% and 100%) identical to SEQ ID NO: 275 or 276.

Other transmembrane domains are known in the art and include CD16, NKG2D, NKp44, NKp46, CD27, DAP10, and DAP12 transmembrane domains.

In some embodiments where the chimeric antigen receptor polypeptide includes a CD3 zeta cytoplasmic signaling domain, the CD3 zeta cytoplasmic signaling domain has an amino acid sequence that is at least 80% (e.g., at least 85%, 90%, 95%, 99% and 100%) identical to SEQ ID NO: 274 (NCBI Reference No: NP_932170) (SEQ ID NO: 274) or a fragment thereof that has activating or stimulatory activity.

As used herein, the term “stimulation” refers to stage of TCR or CAR signaling where a co-stimulatory signal can be used to achieve a robust and sustained TCR or CAR signaling response. As described herein, a co-stimulatory domain can be referred to as a signaling domain. In some cases, a signaling domain (e.g., co-stimulatory domain) can be a CD27, CD28, OX40, CD30, CD40, B7-H3, NKG2C, LIGHT, CD7, CD2, 4-1BB, PD-1, or LFA-1.

In some embodiments where the chimeric antigen receptor polypeptide includes a CD28 co-stimulatory domain, the CD28 co-stimulatory domain is at least 80% (e.g., at least 85%, 90%, 95%, 99% and 100%) identical to SEQ ID NO: 273.

In some embodiments where the chimeric antigen receptor polypeptide includes a OX40 co-stimulatory domain, the OX40 co-stimulatory domain is at least 80% (e.g., at least 85%, 90%, 95%, 99% and 100%) identical to SEQ ID NO: 278.

In some embodiments where the chimeric antigen receptor polypeptide includes a 4-1BB co-stimulatory domain, the 4-1BB co-stimulatory domain is at least 80% (e.g., at least 85%, 90%, 95%, 99% and 100%) identical to SEQ ID NO: 279.

In some embodiments, the first antigen and second antigen are present on CD11c+T-bet+B cells. In some embodiments, the first epitope and the second epitope are on different targets, either on the same cell or same type of cell. In some aspects, the first antigen binding domain is targeted to a first epitope and the second antigen binding domain is targeted to a second epitope. In some embodiments, the first epitope and the second epitope are on the same target, either on the same cell or same type of cells. In some embodiments, the first antigen binding domain and the second antigen binding domain bind the same antigen and the same epitope. In such cases where the first antigen and the second antigen are present on the same cell, the cells is reduced or eliminated as a result of binding of the chimeric antigen receptor.

In some embodiments, the chimeric receptor polypeptide is a chimeric NOTCH receptor polypeptide. In some embodiments, the chimeric NOTCH receptor is a SYNNOTCH® receptor.

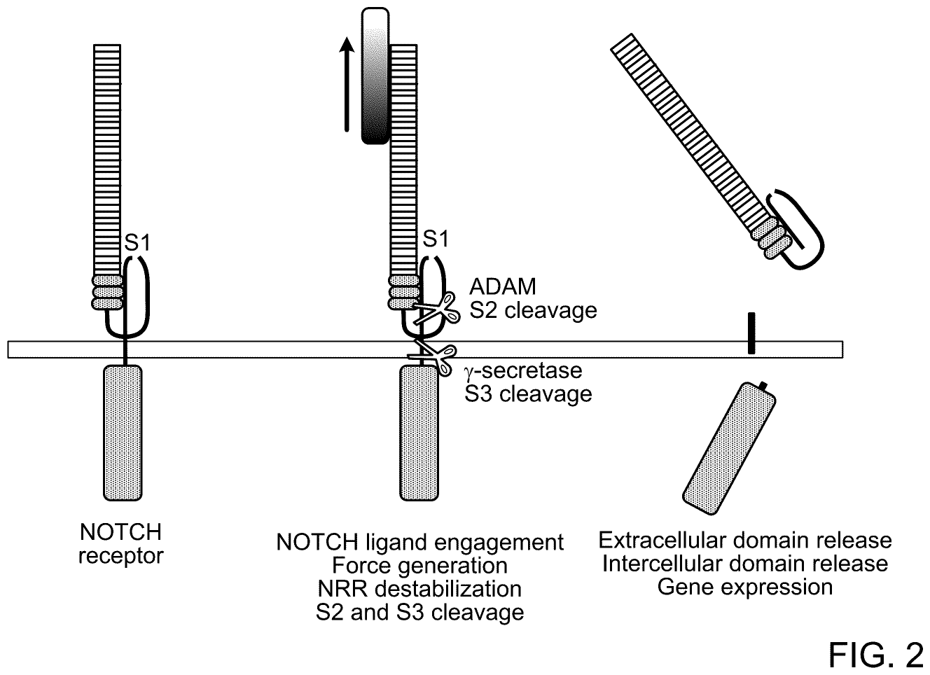

The steps of Notch receptor activation are depicted in FIG. 2. The key structural features of Notch receptors are illustrated, along with the S1 furin cleavage site and S2 ADAM cleavage site. Upon interaction with a Notch ligand, the “pulling” force due to Notch ligand engagement on an adjacent cell unmasks the S2 ADAM cleavage site. S2 cleavage allows S3 gamma-secretase cleavage to occur, resulting in release of the Notch extracellular and intracellular domains (ECD and ICD, respectively). ICD release enables activation of Notch gene expression program. In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence.

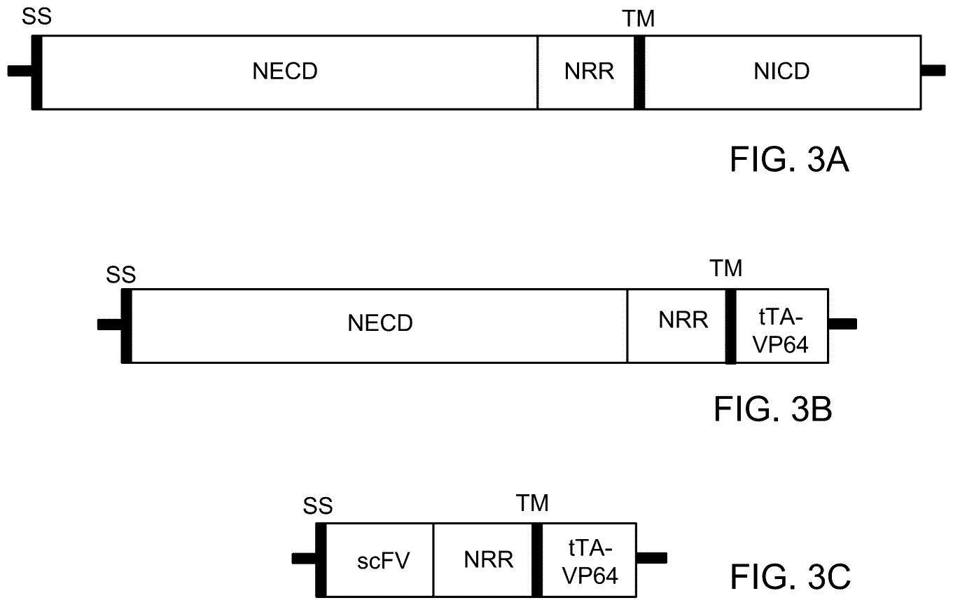

FIGS. 3A-C shows the domain organization of the SYNNOTCH® receptor compared to Notch. SYNNOTCH® is a signal transducing agent derived from Notch receptors by Lim and colleagues. See, U.S. Pat. No. 9,834,608 and Roybal et al., 2016, Cell 167, 419-432, both of which are incorporated by reference in their entireties. SYNNOTCH® was created by isolating the core activation domains of NOTCH, first by replacing the NOTCH EGF domain-rich ligand binding domain with a novel extracellular ligand binding protein to customize the ligand sensing specificity, and secondly, replacing the NOTCH intracellular domain with a customized transcriptional regulator protein domain. Collectively, these two modifications enable the construction of novel chimeric NOTCH receptors that sense customized ligands and induce customized transcriptional programs in the cells that express them. The SYNNOTCH® constructs described below can be constructed as described in U.S. Pat. Nos. 9,670,281 and 9,834,608; and U.S. Patent Applications 20180079812, 20180208636, and 20180355011, each of which is incorporated herein by reference in their entireties.

As used herein are SYNNOTCH® constructs that have substituted for the NOTCH receptor delta binding region, a binding moiety such has an scFV that recognizes an antigen found on B cells, including CD11c+T-bet+B cells, a pan B cell antigen. Upon binding of antigen on CD11c+T-bet+B cells to the scFV on SYNNOTCH®, the NOTCH pathway is activated leading to initiation of transcription of NOTCH downstream target genes. Such target genes can be genes that encode proteins such as growth factors, cytokines, or immunoglobulins, for example.

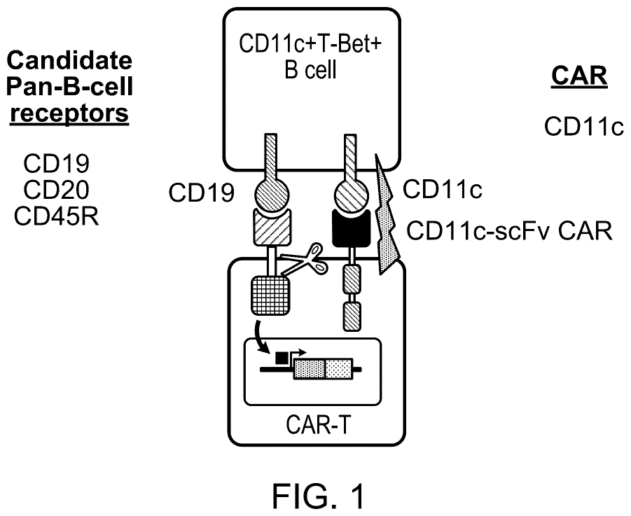

In some embodiments, the NOTCH pathway activation leads to intracellular release of a transcription factor that causes the expression of a chimeric antigen receptor, or CAR, that comprises a second scFv that recognizes a second antigen present on CD11c+T-bet+B cells which reduces or eliminates the CD11c+T-bet+B cells. This second scFv can be, but is not limited to, CD11c. FIG. 1 depicts shows the signal transducing agent, SYNNOTCH®, with several possible pairs of scFvs that can be used to reduce or eliminate CD11c+T-bet+ B cells.

It will be apparent to those skilled in the art that the specificity of the first and second scFvs can be reversed such that the NOTCH pathway activation can occur upon CD11c binding to CD11 c scFv.

In some embodiments, the first antigen binding domain is an antibody or antigen binding fragment. In some embodiments, the antibody binding fragment is selected from the group consisting of a Fab, a F(ab′)2 fragment, a scFv, a scab, a dAb, a single domain heavy chain antibody, and a single domain light chain antibody.

In some embodiments, the first antigen is a B cell receptor. In some embodiments, the B cell receptor the B cell receptor is selected from the group consisting of CD19, CD20, and CD45R.

In some embodiments, the first extracellular binding agent is operably linked to a intracellular domain comprising a proteolytic site and a transcriptional domain such that upon binding of the first antigen the intracellular transcriptional domain is released by proteolysis and relocates to the nucleus where it activates the expression of the CAR.

In some embodiments, the intracellular domain comprises a transcriptional activation domain. In some embodiments, the transcriptional activation domains is selected from the group comprising a VP 16 activation domain, a VP64 activation domain, a p65 activation domain, a MyoD1 activation domain, a Tbx21 activation domain a HSF1 activation domain, a RTA activation domain, a SETT/9 activation domain, a Gal4 DNA binding domain (DBD)-VP64 domain, a tTA-VP64: tetR-VP64 domain, a VP64-p65-Rta (VPR) activation domain, a mini VPR activation domain, a yeast GAL4 activation domain, a yeast HAP1 activation domain, a histone acetyltransferase, or any combination thereof.

In some embodiments where the chimeric antigen receptor polypeptide includes a Tbx21 transcriptional activation domain, the Tbx21 transcriptional activation domain is at least 80% (e.g., at least 85%, 90%, 95%, 99% and 100%) identical to SEQ ID NO: 284.

In some embodiments where the chimeric antigen receptor polypeptide includes a E2S-VP64 transcriptional activation domain, the E2S-VP64 transcriptional activation domain is at least 80% (e.g., at least 85%, 90%, 95%, 99% and 100%) identical to SEQ ID NO: 285.

In some embodiments where the chimeric antigen receptor polypeptide includes a GAL4-VP64 transcriptional activation domain, the GAL4-VP64 transcriptional activation domain is at least 80% (e.g., at least 85%, 90%, 95%, 99% and 100%) identical to SEQ ID NO: 286.

In some embodiments, the first and second extracellular binding agents comprise antibody where the first extracellular binding agent comprises antibody that binds a pan B-cell receptor, and the second extracellular binding agent binds a receptor selectively expressed on CD11c+T-bet+B cells.

In some embodiments, the first and second antigen binding domains comprise an antibody where the first antigen binding domain comprises an antibody that binds a pan B-cell receptor which can be CD19, CD20, or CD45R, and the second antigen binding domain bind receptors selectively expressed on CD11c+T-bet+B cells, which can be CD11c. In some embodiments, expression of the chimeric antigen receptor and binding of the second antigen binding domain to the antigen (e.g., receptors on the surface of the CD11c+T-bet+B cells) selectively expressed on CD11c+Tbet+B cells, the CD11c+Tbet+B cells are reduced or eliminated, thus benefiting a patient suffering from autoimmune disease.

Exemplary CD19 antibodies or antigen binding fragments thereof are described in U.S. Pat. Nos. 11,623,956, 11,618,788, U.S. Patent Publication Number 2023/0099646, U.S. Patent Publication Number 2023/0087263, and U.S. Patent Publication Number 2023/0086030, each of which is incorporated herein by reference in its entirety. Exemplary CD20 antibodies or antigen binding fragments thereof are described in U.S. Pat. Nos. 11,623,005, 11,608,383, 11,603,411, and U.S. Patent Publication No. 2023/0056900, each of which is incorporated herein by reference in its entirety. Exemplary CD45R antibodies or antigen binding fragments thereof are described in U.S. Pat. Nos. 10,093,743, 7,160,987, 6,010,902, each of which is incorporated herein by reference in its entirety.

In various other embodiments, the autoimmune disease can be SLE, rheumatoid arthritis, multiple sclerosis, insulin dependent diabetes mellitus, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, or polyarteritis nodosa.

As used herein, the terms “percent identity” and “identity” in the context of two or more nucleic acids or polypeptides, refer to two or more sequences that are the same or have a specified percentage of nucleotides or amino acid residues that are the same. Percent identity can be determined using sequence comparison software or algorithms or by visual inspection.

In general, percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. The total number of aligned nucleotides or amino acids refers to the minimum number of NOTCH nucleotides or amino acids that are necessary to align the second sequence, and does not include alignment (e.g., forced alignment) with non-NOTCH sequences, such as those fused to NOTCH. The total number of aligned nucleotides or amino acids may correspond to the entire NOTC sequence or may correspond to fragments of the full-length NOTCH sequence.

Sequences can be aligned using the algorithm described by Altschul et al. (Nucleic Acids Res, 25:3389-3402, 1997) as incorporated into BLAST (basic local alignment search tool) programs, available at ncbi.nlm.nih.gov on the World Wide Web. BLAST searches or alignments can be performed to determine percent sequence identity between a NOTCH nucleic acid or polypeptide and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a NOTCH sequence and another sequence, the default parameters of the respective programs are used.

As used herein, the term “antibody” can refer to an intact immunoglobulin or to an antigen binding portion thereof. Antigen binding portions can be produced by recombinant DNA techniques or by enzymatic or chemical cleavage of intact antibodies. Examples of antigen binding portions include Fab, Fab′, F(ab′)₂, Fv, domain antibodies (dAbs), complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide. scFv antibody fragments include the V_{H} and V_{L} domains of an antibody, where the domains are present in a single polypeptide chain. In some cases, an antibody can be a human or humanized antibody.

The term “affinity” as used herein, refers to the binding of mutant porcine IL-2 to the human IL-2 receptor, trimeric or dimeric forms. Affinity can be measured using any suitable method. See, e.g., Shanafelt et al., 2000 Nature Biotechnol 18: 1197-1202.

The term “substantially purified” as used herein refers to a protein (or the polynucleotide encoding the protein) that has been separated from biological components such that a substantially pure protein (or polynucleotide) will comprise at least 85% of a sample.

### EXAMPLES

The following examples are intended to provide a description of how to make and use the present invention. The examples are not, however, intended to limit the scope of what the inventors regard as their invention, nor are they intended to suggest that the experiments are all the experiments that can be performed. Efforts have been made to ensure accuracy with respect to numbers used (e.g. amounts, temperature, etc.) but some experimental errors and deviations are standard in the art and known to the skilled practitioner.

### Example 1

### Construction of Chimeric Receptor Polypeptides and Chimeric Antigen Receptors

Construction of Signal Transducing Agent with CD19 scFv and CD11c scFv CAR, or CD19 scFv and B220 scFv.

The signal transducing agent, SYNNOTCH®, is described in U.S. Pat. Nos. 9,670,281 and 9,834,608; and U.S. Patent Applications 20180079812, 20180208636, and 20180355011. The materials and methods for construction of SYNNOTCH® Receptors with appropriate response elements are described in U.S. Pat. Nos. 9,670,281 and 9,834,608, and pending applications, above. Each of the aforementioned patents and applications are incorporated herein by reference in their entireties.

### SYNNOTCH® Chimeric Receptors and Anti-CD11c CAR Design.

SYNNOTCH® constructs are designed in which a SYNNOTCH® receptor for pan-human B cell antigens such as CD19, CD20, or B220 (CD45R, See, Rodig et al., Hum Pathol 2005, 36(1):51), drives the inducible expression of a CAR for anti-human CD11c antigen. As described in U.S. Pat. Nos., 9,670,281 and 9,834,608, an anti-CD19 SYNNOTCH® receptor is constructed by fusing an anti-human CD19 scFv to the Notchl core intracellular domain and Gal4 DNA binding domain (DBD)-VP64 fusion. The expression of Gal4VP64 protein turns on the multimerized transcriptional response element (TRE) regulating an anti-human CD11c CAR, which is composed of the anti-CD11c scFv and CD8 alpha hinge region as the extracellular domain, and CD28-4-1BB and CD3zeta as the intracellular signaling domain. Similarly, an anti-B220 SYNNOTCH® receptor targeting human B220 is constructed by replacement of the CD19 scFv with mouse/human cross-reactive B220 scFv.

### Example 2

### Generation of CAR-T Cells Having Chimeric Receptor Polypeptides and Chimeric Antigen Receptors

### Generation of SYNNOTCH-CAR T Cells

Primary human T cell isolation, and culture and lentiviral transduction of human T cells are performed as described elsewhere (see, e.g., U.S. Pat. Nos. 9,670,281 and 9,834,608). Briefly, purified human T cells from healthy blood donors are lentivirally transduced with either anti-CD19 or anti-B220 SYNNOTCH® receptor (Myc-tagged) and a TRE-inducible promoter controlling expression of the anti-CD11c CAR (Flag-tag). After lentiviral transduction, the expression of both the SYNNOTCH® receptor and the inducible anti-CD11c CAR are assessed by staining with anti-myc-tag Alexa Fluor647 (Cell Signaling #2233) and anti-flag Alexa Fluor488 (Cell Signaling #15008).

### Generation of Target Cell Lines Expressing Human CD19 and CD11c

To evaluate the ability of SYNNOTCH® constructs generated as described above to kill cells expressing CD11c, human K562 cell line and human B cell lines such as Nalm6, Raji or Daudi are used to generate stable target cell clones as described elsewhere (Ellebrecht et al., Science 353:179-184, 2016). Briefly, either K562 or B cell lines are lentivirally transduced with an expression construct containing human CD11c cDNA, a constitutive cassette driving GFP expression and a drug selection gene (e.g., hygromycin or puromycin). After lentiviral transduction, CD11c-expressing B cells are single-cell cloned by limiting dilution based on their GFP expression. See, e.g., Freshney, (2010), Culture of Animal Cells: A Manual of Basic Technique and Specialized Applications (6th edition) Hoboken, N. J.: Wiley-Blackwell. pp. 208-211.

### Characterization and Functional Analysis of SYNNOTCH-CAR T Cells

Cytotoxicity T lymphocyte assay: SYNNOTCH®-CAR T cells (effectors) and engineered target cell lines (targets) expressing both CD19/B220 and CD11c proteins are co-cultured at 37° C. overnight at various effector and target (E:T) ratios. Cells are harvested and lactose dehydrogenase (LDH), a stable cytosolic enzyme that is released upon cell lysis, is measured using a bioluminescence-based LDH-Glo cytotoxicity Assay Kit (Promega, Cat#: J2380). In addition to engineered target cell lines, human ABCs from elderly female patients with lupus are used to evaluate the cytotoxicity of the engineered SYNNOTCH®-CAR T cells. First, human ABCs from patients are isolated by magnetic cell sorting system (see, e.g., Miltenyi et al., Cytometry 11:231-238, 1990). Second, SYNNOTCH®—CAR T cells are co-cultured with purified human ABCs at 37° C. as described above, using engineered target cell lines. Third, after three days of co-culture, the efficiency of cell killing is assessed by the percentage of ABC cell survival using surface staining with anti-CD19 and anti-CD11c antibodies (Rubtsov et al., supra) or by LDH-Glo cytotoxicity Assay (Promega; Madison, WI).

SYNNOTCH®-CAR T cells are stimulated at 37° C. overnight with target cells lines as described elsewhere (see, Roybal et al., Cell 164:770-779, 2016). The supernatant from the co-culture is analyzed for the presence of cytokines such as IFNgamma or IL-2 by ELISA assay (e.g., R&D Systems; cat #: DIF50 for human IFNgamma ELISA kit and cat #:D2050 human IL-2 ELISA kit).

### Example 3

### Binding Kinetics of CD19 and CD11c scFvs

Table 1 below shows the binding kinetics of scFvs binding either CD19 or CD11c. The data demonstrate functional antigen-binding fragments capable of specifically binding CD19 or CD11c.

TABLE 1

| Name | Target | SEQ ID NO | Kd |
|---|---|---|---|
| 2A07 | CD19 | SEQ ID NO: 1 | 6.26E−09 |
| 2G08 | CD19 | SEQ ID NO: 2 | 1.25E−07 |
| 3E09 | CD19 | SEQ ID NO: 3 | 5.99E−09 |
| 2E03 | CD19 | SEQ ID NO: 4 | 9.52E−09 |
| 2A05 | CD19 | SEQ ID NO: 5 | 6.90E−09 |
| 1A01 | CD19 | SEQ ID NO: 6 | 6.21E−09 |
| 1A11 | CD19 | SEQ ID NO: 7 | 8.78E−09 |
| 1E09 | CD19 | SEQ ID NO: 8 | 4.55E−09 |
| 3B04 | CD19 | SEQ ID NO: 9 | 6.24E−09 |
| 2E05 | CD19 | SEQ ID NO: 10 | 8.14E−09 |
| 3.9 | CD11c | SEQ ID NO: 37 | 7.54E−08 |
| 3.9 | CD11c | SEQ ID NO: 38 | 1.19E−08 |

### Example 4

### Treatment of Systemic Lupus Erythematosus (SLE)

A mammal suffering from SLE will be assayed to determine the presence of SLE autoantibodies prior to and after treatment with an immuno-activatable T cell generated in Example s. Human ABCs from peripheral blood samples of SLE patients can be isolated by magnetic cell sorting system (Miltenyi et al., Cytometry 11: p231-238 (1990)). The isolated cells are then stimulated for 5-7 days with CD40L (R &D system, Cat# 6420-CL) and CpG ODN 2006 (InvivoGen, Cat# tlrl-2006) in the presence or absence immuno-activatable T cell comprising a CD19 CAR a CD11c CAR. Culture supernatants will be tested for secreted Immunoglobulins (e.g., IgM, IgA and IgG) by ELISA assay and ANA autoantibodies by immunofluorescence analysis as described previously (Capolunghi et al., Rheumatology 49: p2281-89 (2010)). A patient is administered a dose of the immuno-activatable T cell comprising the CD19 CAR and the CD11c CAR, in the range of 10-100 million T cells. The dose will be empirically determined depending on a number of factors, including side effects, and indications of efficacy. The modified T-cells can be administered by any method known in the art including, without limitation, intravenous, subcutaneous, intranodal, intratumoral, intrathecal, intrapleural, intraperitoneal and directly to the thymus. A single dose or multiple doses may be administered.

SEQUENCE APPENDIX
SEQ ID NO: 1 2A07 scFv
EVQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARYIQGLGYYFDYWGQGTLVTVSTGGGGSGGGGSGGGGSSYELMQPPSVS
VSPGQTASITCSGDKLGDKYVSWYQQKPGQSPVLVIYQDTKRPSGIPERFSGSNSGNTATLTISGTQAMDEAD
YYCQAWDSGTAIFGGGTKVTVL
SEQ ID NO: 2 2G08 scFv
QVQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARYIQGLGYYFDYWGQGTLVTVSTGGGGSGGGGSGGGGSQAGLTQPPSVS
VSPGQTASITCSGDKLGDKYVSWYQQKPGQSPVLVIYQDSKRPSGIPERFSGSNSGNTATLTISGTQAMDEAD
YYCQAWDSSTVVFGGGTKVTVL
SEQ ID NO: 3 3000000000 scFv
QLQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARYIQGLGYYFDYWGQGTLVTVSTGGGGSGGGGSGGGGSQAGLTQPPSVS
VSPGQTASITCFGDKLGHKYVSWYQQKPGQSPVLVIYQDSKRPSGIPERFSGSNSGNTATLTISGTQAMDEAD
YYCQAWDSSTVVFGGGTKLTVL
SEQ ID NO: 4 ′2E03 scFv
QMQLVQSGGGVVQPGGSLRLSCAASGFTFSSYGMHWVRQAPGKGLEWVAFIRYDGSNKYYADSVKGRFTISRD
NSKNTLYLQMNSLRAEDTAVYYCAKPSRGYSRSLDYWGQGTLVTVSTGGGGSGGGGSGGGGSQPVLTQPPSVS
VSPGQTASITCSGDKLGDKFTSWYQQRPGQSPVLVIYQDNKRPSGIPERFSGSNSGNTATLTISRVEAGDEAD
YYCQVWDSSSDHWVFGGGTQLTVL
SEQ ID NO: 5 2A05 scFv
QVQLVESGAEVKKPGESLKISCKGSGYSFTSYWIGWVRQMPGKGLEWMGIIYPGDSDTRYSPSFQGQVTISAD
KSISTAYLQWSSLKASDTAMYYCARLQSGWLHAFDIWGQGTMVTVSTGGGGSGGGGSGGGGSQSVLTQPPSVS
VSPGQTARISCSGDKLGDKYVSWYQQKPGQSPVLVIYEDSKRPSGIPERLSGSNSGNTATLTISGTQAMDEAD
YYCQAWDSSTVVFGGGTKLTVL
SEQ ID NO: 6 1A01 scFv
QVQLLQSAAEVKKPGESLKISCKGSGYSFTSYWIGWVRQMPGKGLEWMGIIYPGDSDTRYSPSFQGQITISAD
KSISTAYLQWSSLKASDTAMYYCARLKWSGLSHYYYYYMDVWGKGTTVTVSTGGGGSGGGGSGGGGSLSELTQ
DPAVSVALGQTVRITCQGDSLRNYYASWYQQKPGQAPVLVIYGKNNRPSGIPDRFSGSRSGNTASLTITGAQA
EDEADYYCNSRDSSGNHPVVFGGGTKLTVL
SEQ ID NO: 7 1A11 scFv
QVQLLQSAAEVKKPGESLKISCKGSGYSFTSYWIGWVRQMPGKGLEWMGIIYPGDSDTRYSPSFQGQVTISAD
KSISTAYLQWSSLKASDTAMYYCARLKWSGLSHYYYYYMDVWGKGTTVTVSTGGGGSGGGGSGGGGSLSELTQ
DPAVSVALGQTVRITCQGDSLRSYYASWYQQKPGQAPVLVIYGKNNRPSGIPDRFSGSSSGNTASLTITGAQA
EDEADYYCNSRDSSGNHLVFGGGTKLTVL
SEQ ID NO: 8 1000000000 scFv
QVQLLQSAAEVKKPGESLKISCKGSGYSFTSYWIGWVRQMPGKGLEWMGIIYPGDSDTRYSPSFQGQVTISAD
KSISTAYLQWSSLKASDTAMYYCARLKWSGLSHYYYYYMDVWGKGTTVTVSTGGGGSGGGGSGGGGSLSELTQ
DPAVSVALGQTVRITCQGDSLRSYYASWYQQKPGQAPVLVIYGKNNRPSGIPDRFSGSSSGNTASLTITGAQA
EDEADYYCNSRDSSGNHVIFGGGTKLTVL
SEQ ID NO: 9 3B04 scFv
QVQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARLPLGLQVGFDYWGQGTLVTVSTGGGGSGGGGSGGGGSLPVLTQPPSVS
VSPGQTASITCSGDKLGDKYASWYQQKPGQSPVLIIYQDTKRASGIPERFSGSNSGNTATLTISGTQAVDEAD
YYCQAFDSSAAHFVFGAGTKLTVL
SEQ ID NO: 10 200000 scFv
QMQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARVRYSYDLNFDYWGQGTLVTVSTGGGGSGGGGSGGGGSSYELMQPPSVS
VSPGQTASITCSGDKLGDKYASWYQQKPGQSPVLVIYQDNKRPSGIPERFSGSNSGNTATLTISGTQAMDEAD
YYCQTWDSSTAVFGGGTKVTVL
SEQ ID NO: 11 2B2 scFv
QVQLQQSGAELAKPGASVKLSCKTSGYTFTNFWMHWVKQRPGQGLEWIGYINPSSDYTKYNQKFKGKATLTAD
KSSSTAYMQLSSLTYEDSAVYYCARDDYSDFGFAYWGQGTLVTVSAGGGGSGGGGSGGGGSDIQMTQSPASLS
ASVGETVTITCRASENIYSFLAWYQQKQGKSPQLLVYNAKTLAEGVPSRFSGSGSGTQFSLKINSLQPEDFGS
YYCQHHYGIPPTFGGGTKLEIK
SEQ ID NO: 12 3D4-LC1 scFv
QVQLQQSGAELARPGASVKMSCKASGYTFTSYTMHWVKQRPGQGLEWIGYINPSSGYTKYNQKFKDKATLTAD
KSSSTAYMQLSSLTSEDSAVYYCAREANWDDVDYWGQGTTLTVSSGGGGSGGGGSGGGGSQIVLTQSPAIMSA
SPGEKVTMTCSASSSVSYMYWYLQKPGSSPRLLIYDTSNLASGVPVRFSGSGSGTSYSLTISRMEAEDAATYY
CQQWSSYPLTFGAGTKLELK
SEQ ID NO: 13 3D4-LC2 scFv
QVQLQQSGAELARPGASVKMSCKASGYTFTSYTMHWVKQRPGQGLEWIGYINPSSGYTKYNQKFKDKATLTAD
KSSSTAYMQLSSLTSEDSAVYYCAREANWDDVDYWGQGTTLTVSSGGGGSGGGGSGGGGSDVLMTQTPLSLPV
SLGDQASISCRSSQSIVHSNGNTYLEWYLQKPGQSPKLLIYKVSNRFSGVPDRFSGSGSGTDFTLKISRVEAE
DLGLYYCFQGSHVPYTFGGGTKLEIK
SEQ ID NO: 14 4A5 scFv
EFQLQQSGPELVKPGASVKISCKASGYSFTDYNMNWVKQSNGKSLEWIGVINPNYGTTSYNQKFKGKATLTVD
QSSSTAYMQLNSLTSEDSAVYYCARNYYSSSYDGYFDYWGQGTTLTVSSGGGGSGGGGSGGGGSDIVLTQSPA
SLAVSLGQRATISCRASESVDNYGISFMHWYQQKPGQPPKFLIYRASNLESGIPARFSGSGSRTDFTLTINPV
ETDDVATYYCQQSNKDPRTFGGGTKLEIK
SEQ ID NO: 15 400000000 scFv
QVQLQQSGPELVKPGASVKISCRASGYTFTDYYIDWVKQRPGQGLEWIGWIFPGTNSTYYNEKFKGKATLTVD
KSSSTAYMLLSSLTSEDSAVYFCARSGLRDFDYWGQGTTLTVSSGGGGSGGGGSGGGGSDIVMTQSPATLSVT
PGDRVSLSCRASQSISDYLHWYQQKSHESPRLLIKYASQSISGIPSRFSGSGSGSDFTLSINSVEPEDVGVYY
CQNGHSFPLTFGAGTKLELK
SEQ ID NO: 16 4G6 scFv
EVQLQQSGPVLVKPGASVKMSCKASGYTFTDYYMNWVKQSHGKGLEWIAVINPYSGGTSYNQKFKGKATLTVD
KSSSTAYMELSSLTSEDSAVYYCASVSSYGNYFDYWGQGTTLTVSSGGGGSGGGGSGGGGSDIVLTQSPASLA
VSLGQRATISCRASESVSIHASHLLHWYQQKPGQPPKLLIYAASNLESGVPARFSGSGSETDFTLNIHPVEEE
DAATYFCQQSIEDPWTFGGGTKLEIK
SEQ ID NO: 17 500 scFv
QVQLQQSGPELVKPGASVKISCKASGYTFTDYYINWVKQRPGQGLEWIGWIFPGSGSTYYNEKFKGKATLTVD
KSSSTVYMLLSSLTSEDSAVYFCAREAKLGRDYFDYWGQGTTLTVSSGGGGSGGGGSGGGGSDIVMTQSHKFM
STSVGDRVSITCKASQDVSTAVAWCQQKPGQSPKLLIYSASYRYTGVPDRFTGSGSGTDFTFTISSVQAEDLA
VYYCQQHYSTPYTFGGGTRLEIK
SEQ ID NO: 18 5G7 scFv
EFQLQQSGPELVKPGASVKISCKASGYSFTDYNMNWVKQSNGKSLAWIGVINPNYGTTNYNQKFKGKATLTVD
QSSSTAYMQLNSLTSEDSAVYYCARNYYGSTYDGYFDYWGQGTTLTVSSGGGGSGGGGSGGGGSDIVLTQSPA
SLAVSLGQRATISCRASESVDNYGISFMHWYQQKPGQPPKFLIYRASNLESGIPARFSGSGSRTDFTLTINPV
ETDDVATYYCQQSNKDPRTFGGGTKLEIT
SEQ ID NO: 19 5H1 scFv
EFQLQQSGPELVKPGASVKISCKASGYSFTDYNMNWVKQSNGKSLAWIGVINPNYGTTNYNQKFKGKATLTVD
QSSSTAYMQLNSLTSEDSAVYYCARNYYGSTYDGYFDYWGQGTTLTVSSGGGGSGGGGSGGGGSDIVLTQSPA
SLAVSLGQRATISCRASESVDNYGISFMHWYQQKPGQPPKFLIYRASNLESGIPARFSGSGSRTDFTLTINPV
ETDDVATYYCQQSNKDPRTFGGGTKLEIT
SEQ ID NO: 20 6B2 scFv
QVQLQQSGAELMKPGASVKISCKATGYTINGYWIEWVKERPGHGLEWIGEILPGSGSTNYNEKFKGKATFTAD
TSSNTAYMQLSSLTTEDSAIYYCARGMEGAMDYWGQGTSVTVSSGGGGSGGGGSGGGGSDIVMTQSHKFMSTS
VGDRVSITCKASQDVSTAVAWYQKKPGQSPKLLIYSASYRYTGVPDRFTGSGSGTDFTFTISSVQAEDLAVYY
CQQHYSTPPTFGGGTKLEIK
SEQ ID NO: 21 6B3 scFv
QVQLQQPGAELVMPGASVRLSCKASGYTFTSYWMHWVKQRPGQGLEWIGEIDPSESYPNYNQNFKGKATLTVD
KSSSTAYMQLSSLTSEDSAVYYCARSYYGRSGYAMDYWGQGTSVTVSSGGGGSGGGGSGGGGSNIVMTQSPKS
TSMSVGERVTLNCKASENVGTYVSWYQQKPEQSPKLLIYGASNRYTGVPDRFTGSGSATDFTLTISSVQAEDL
ADYHCGQSYSYPPFTFGSGTKLEIK
SEQ ID NO: 22 600000 scFv
QVQLTESGPGLVAPSQSLSITCTVSGFSLTNYIISWVRQPPGKGLEWLGVIWTGGGTNYNSALKSRLSISKDD
SKSQVFLKMNSLQTDDTARYYCARNEAVVAIFDWYFDVWGTGTTVTVSSGGGGSGGGGSGGGGSDIQMTQTTS
SLSASLGDRVTISCRASQDISNYLNWYQQKPDGAVKLLIYYTSRLHSGVPSRFSGSGSGTDFSLTISNLEQED
FATYFCQQGNTLPWTFGGGTKLEIK
SEQ ID NO: 23 6F3 scFv
EFQLQQSGPELVKPGASVKISCKASGYSFTDYNMNWVKQSNGKSLEWIGVINPNYGTTSYNQKFKGKATLTVD
QSSSTAYMQLNSLTSEDSAVYYCARNYYGNNYDGYFDYWGQGTTLTVSSGGGGSGGGGSGGGGSDIVLTQSPA
SLAVSLGQRATISCRASESVDNYGISFMHWYQQKPGQPPKFLIYRASNLESGIPARFSGSGSRTDFTLTINPV
ETDDVATYYCQQSNKDPRTFGGGTKLEIT
SEQ ID NO: 24 6F4 scFv
QIQLVQSGPELKKPGETVKISCKASGYTFTMYGMSWVKQAPGKGLKWMGWINTYSGVPTYADDFKGRFAFSLE
TSANTAYLQINNLKNEDTATYFCARFPYDYDGYFDVWGTGTTVTVSSGGGGSGGGGSGGGGSDIVMTQSHKFM
STSVGDRVSITCKASQDVGTAVAWYQQKPGQSPKLLIYWASTRHTGVPDRFTGSGSGTDFTLAISNVQSEDLA
DYFCHQFSSYPLTFGAGTRLELK
SEQ ID NO: 25 9B3 scFv
EVQLQQSGPELVKPGASVKISCKASGYTFTDYYMNWVKQSHGKSLEWIGDINPNNGGTTYNQKFKGKATLTVD
KSSSTAYMELRSLTSEDSAVYYCARRYYSSGYDGYFDVWGTGTTVTVSSGGGGSGGGGSGGGGSDIVLTQSPA
SLAVSLGQRATISCRASENVDNYGISFMHWYQQKPGQPPKFLIYRASNLEYGIPARFSGSGSRTDFTLTINPV
ETDDVATYYCQQSNKDPLTFGAGTKLELK
SEQ ID NO: 26 15H3 scFv
EVQLQQSGPELVKPGASVKMSCKASGSTFTSYVMHWVKQKPGQGLEWIGYSNPYNDGTKYNEKFKGKATLTSD
KSSSTAYMELSSLTSEDSAVYYCARLNVLYYFDNWGQGTTLTVSSGGGGSGGGGSGGGGSDIVLTQSPASLAV
SLGQRATISCRASKSVSTSGYTYMHWYQQKPGQPPKLLIYLASNLESGVPARFSGSGSGTDFTLNIHPVEEED
AATYYCQHSRELPLTFGAGTKLELK
SEQ ID NO: 27 8C3 scFv
QVQLQQPGTELVKPGASVKLSCKASGYTFTSYWMHWVKQRPGQGLEWIGNINPGNGGTDYNEKIKSKATLTVD
KSTSTAYMQLSSLTSEDSAVYYCARGGGYYGYDGYWYFDVWGTGTTVTVSSGGGGSGGGGSGGGGSDIVMTQA
AFSIPVTLGTSTSISCRSTKSLLHSNGITYLYWYLQKPGQSPQLLIYQMSNLASGVPDRFSSSGSGTDFTLRI
SRVEAEDVGVYYCAQNLELPWTFGGGTKLEIK
SEQ ID NO: 28 4C7 scFv
EVQLQQSGPVLVKPGPSVKISCEASGFTFTDYYMHWVKQNHGKSLEWIGLVYPYNGDTIYNQKFKGKATLTVD
TSSSTAYMDLHSLTSEDSAVYYCARGANWGDYWGQGTTLTVSSGGGGSGGGGSGGGGSDVVMTQTPLSLPVSL
GDQASISCRSSQSLVHSNGNTYLHWFLQKPGQSPKLLIYKVSNRFSGVPDRFSGSGSGTDFTLKISRVEAEDL
GLYFCSQSTHVPPTFGGGTKLEIK
SEQ ID NO: 29 4D4 scFv
QVQLQQPGTELVKPGASVKLSCKASGYTFTSYWMHWVKQRPGQGLEWIGNISPSNGGTNYNENFKSKATLTVD
KSSSTAYMQLSSLTSEDSAVYYCATYYVDYWGQGTTLTVSSGGGGSGGGGSGGGGSDVVMTQTPLTLSVTIGQ
PASISCKSTQSLLDSDGKTYLNWFLQRPGQSPKRLIYLVSKLDSGVPDRFTGSGSGTDFTLKISRVEAEDLGL
YYCWQGTHFPQTFGGGTKLEIK
SEQ ID NO: 30 6A3 scFv
QVTLKESGPGILQSSQTLSLTCSFSGFSLSTSGMGVSWIRQPSGKGLEWLAHIYWDDDKRYNPSLKSRLTISK
DTSRNQVFLKITSVDTADTATYYCARRVYGYDPYAMNYWGPGTSVTVSSGGGGSGGGGSGGGGSQIVLTQSPA
LMSASPGEKVTMTCSASSSVSYMYWYQQKPRSSPKPWIYLTSTLASGVPARFSGSGSGTSYSLTISSMEAEDA
ATYYCQQWSSNPYTFGGGTKLEIK
SEQ ID NO: 31 8B8 scFv
EVQLQQSGPELVKPGASVKISCKASGYTFTDYYMNWVKQSHGKSLEWIGDINPNNGGTSYNQKFKGKATLTVD
KSSSTAYMELRSLTSEDSAVYYCAPHYYGSSYDWYFDVWGTGTTVTVSSGGGGSGGGGSGGGGSDIVLTQSPA
SLAVSLGQRATISCRASESVDNYGISFMHWYQQKPGQPPKLLIYRASNLESGIPARFSGSGSRTDFTLTINPV
ETDDVATYYCQQSNKDPLTFGAGTKLELK
SEQ ID NO: 32 9B1 scFv
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCAREDDSRYWYFDVWGTGTTVTVSSGGGGSGGGGSGGGGSDIVMTQSHKFM
STSVGDRVSITCKASQDVGTAVAWYQQKPGQSPKLLIYWASTRHTGVPDRFTGSGSGTDFTLTISNVQSEDLA
DYFCQQYSSYPYTFGGGTKLEIK
SEQ ID NO: 33 9B2 scFv
QIQFVQSGPELKKPGETVKISCKASVYTFTEYPMHWVKQAPGKGFKWMGWINTYSGEPTYADDFKGRFAFSLE
TSASTAYLQINNLKNEDTASYFCAREGWLDAMDYWGQGTSVTVSSGGGGSGGGGSGGGGSDIVMTQSQKFMST
IVGDRVSITCKASQNVGTAVAWYQQKPGQSPKLLIYSASNRYTGVPDRFTGSGSGTDFTLTISNMQSEDLADY
FCQQYSSYTWTFGGGTKLEIK
SEQ ID NO: 34 9C1 scFv
QVQLKQSGPGLVQPSQSLSITCTVSGFSLTTFGVHWVRQSPGKGLEWLGVIWSGGSTDYNAAFISRLSISKDN
SKSQVFFKMNSLQVDDTAIYYCAPRLGLRAYWGQGTLVTVSAGGGGSGGGGSGGGGSDIKMTQSPSSMYASLG
ERVTITCKASQDINSYLSWFQQKPGKSPKTLIYRANRLVDGVPSRFSGSGSGQDYSLTISSLEYEDMGIYYCL
QYDEFPYTFGGGTKLEVK
SEQ ID NO: 35 9G1 scFv
QVQLKQSGPGLVQPSQSLSITCTVSGFSLTSYGVHWVRQSPGKGLEWLGVIWSGGTTDYNAAFISRLSISKDN
SKSQVFFKMNSLQADDTAIYYCARMGGTGYFDVWGTGTTVTVSSGGGGSGGGGSGGGGSNIVMTQSPKSMSMS
VGERVTLSCKAGENVGPYVSWYQQKPEQSPKLLIYGASNRFTGVPDRFTGSGSATDFTLTISSVQAEDLADYH
CGQSYSYPFTFGSGTKLEIK
SEQ ID NO: 36 3G6 scFv
QVQLKQSGPGLVQPSQSLSITCTVSGFSLTSYGVHWVRQSPGKGLEWLGVIWSGGTTDYNAAFISRLSISKDN
SKSQVFFKMNSLQADDTAIYYCARMGGTGYFDVWGTGTTVTVSSGGGGSGGGGSGGGGSDIVMTQAAFSNPVT
LGTSASISCRSSKSLLHSNGITYLYWYLQKPGLSPQLLIYHMSNLASGVPDRFSSSGSGTDFTLRISRVEAED
VGVYYCAQNLELPWTFGGGTKLEIK
SEQ ID NO: 37 3.9 VH-VL full scFv
QVQLVQSGAEVKKPGASVKVSCKASGYTFTDYNMHWVRQAPGQGLEWMGYIYPYNGGTAYNQKFKNRVTMTRD
TSTSTVYMELSSLRSEDTAVYYCARGLDVMDYWGQGTLVTVSSGGGGSGGGGSGGGGSAIQLTQSPSSLSASV
GDRVTITCRASENIYSYLAWYQQKPGKAPKLLIYNAKILAEGVPSRFSGSGSGTDFTLTISSLOPEDFATYYC
QHHYVSPRTFGQGTKLEIK
SEQ ID NO: 38 VL-VH full scFv
DIQMTQSPSSLSASVGDRVTITCRASENIYSYLAWYQQKPGKAPKLLIYNAKILAEGVPSRFSGSGSGTDFTL
TISSLQPEDFATYYCQHHYVSPRTFGQGTKLEIKGGGGSGGGGSGGGGSQVQLVQSGAEVKKPGASVKVSCKV
SGYTFTDYNMHWVRQAPGKGLEWMGYIYPYNGGTAYNQKFKNRVTMTEDTSTDTAYMELSSLRSEDTAVYYCA
RGLDVMDYWGQGTLVTVSS
SEQ ID NO: 39 2A07 VH Domain
EVQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARYIQGLGYYFDYWGQGTLVTVST
SEQ ID NO: 40 2G08 VH Domain
QVQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARYIQGLGYYFDYWGQGTLVTVST
SEQ ID NO: 41 3000000000 VH Domain
QLQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARYIQGLGYYFDYWGQGTLVTVST
SEQ ID NO: 42 ′2E03 VH Domain
QMQLVQSGGGVVQPGGSLRLSCAASGFTFSSYGMHWVRQAPGKGLEWVAFIRYDGSNKYYADSVKGRFTISRD
NSKNTLYLQMNSLRAEDTAVYYCAKPSRGYSRSLDYWGQGTLVTVST
SEQ ID NO: 43 2A05 VH Domain
QVQLVESGAEVKKPGESLKISCKGSGYSFTSYWIGWVRQMPGKGLEWMGIIYPGDSDTRYSPSFQGQVTISAD
KSISTAYLQWSSLKASDTAMYYCARLQSGWLHAFDIWGQGTMVTVST
SEQ ID NO: 44 1A01 VH Domain
QVQLLQSAAEVKKPGESLKISCKGSGYSFTSYWIGWVRQMPGKGLEWMGIIYPGDSDTRYSPSFQGQITISAD
KSISTAYLQWSSLKASDTAMYYCARLKWSGLSHYYYYYMDVWGKGTTVTVST
SEQ ID NO: 45 1A11 VH Domain
QVQLLQSAAEVKKPGESLKISCKGSGYSFTSYWIGWVRQMPGKGLEWMGIIYPGDSDTRYSPSFQGQVTISAD
KSISTAYLQWSSLKASDTAMYYCARLKWSGLSHYYYYYMDVWGKGTTVTVST
SEQ ID NO: 46 1000000000 VH Domain
QVQLLQSAAEVKKPGESLKISCKGSGYSFTSYWIGWVRQMPGKGLEWMGIIYPGDSDTRYSPSFQGQVTISAD
KSISTAYLQWSSLKASDTAMYYCARLKWSGLSHYYYYYMDVWGKGTTVTVST
SEQ ID NO: 47 3B04 VH Domain
QVQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARLPLGLQVGFDYWGQGTLVTVST
SEQ ID NO: 48 200000 VH Domain
QMQLVQSGAEVKKPGESLKISCKGSGYSFTSYWIGWVRQMPGKGLEWMGIIYPGDSDTRYSPSFQGQVTISAD
KSISTAYLQWSSLKASDTAMYYCARVRYSYDLNFDYWGQGTLVTVST
SEQ ID NO: 49 2B2 VH Domain
QVQLQQSGAELAKPGASVKLSCKTSGYTFTNFWMHWVKQRPGQGLEWIGYINPSSDYTKYNQKFKGKATLTAD
KSSSTAYMQLSSLTYEDSAVYYCARDDYSDFGFAYWGQGTLVTVSA
SEQ ID NO: 50 3D4-LC1 VH Domain
QVQLQQSGAELARPGASVKMSCKASGYTFTSYTMHWVKQRPGQGLEWIGYINPSSGYTKYNQKFKDKATLTAD
KSSSTAYMQLSSLTSEDSAVYYCAREANWDDVDYWGQGTTLTVSS
SEQ ID NO: 51 3D4-LC2 VH Domain
QVQLQQSGAELARPGASVKMSCKASGYTFTSYTMHWVKQRPGQGLEWIGYINPSSGYTKYNQKFKDKATLTAD
KSSSTAYMQLSSLTSEDSAVYYCAREANWDDVDYWGQGTTLTVSS
SEQ ID NO: 52 4A5 VH Domain
EFQLQQSGPELVKPGASVKISCKASGYSFTDYNMNWVKQSNGKSLEWIGVINPNYGTTSYNQKFKGKATLTVD
QSSSTAYMQLNSLTSEDSAVYYCARNYYSSSYDGYFDYWGQGTTLTVSS
SEQ ID NO: 53 400000000 VH Domain
QVQLQQSGPELVKPGASVKISCRASGYTFTDYYIDWVKQRPGQGLEWIGWIFPGTNSTYYNEKFKGKATLTVD
KSSSTAYMLLSSLTSEDSAVYFCARSGLRDFDYWGQGTTLTVSS
SEQ ID NO: 54 4G6 VH Domain
EVQLQQSGPVLVKPGASVKMSCKASGYTFTDYYMNWVKQSHGKGLEWIAVINPYSGGTSYNQKFKGKATLTVD
KSSSTAYMELSSLTSEDSAVYYCASVSSYGNYFDYWGQGTTLTVSS
SEQ ID NO: 55 500 VH Domain
QVQLQQSGPELVKPGASVKISCKASGYTFTDYYINWVKQRPGQGLEWIGWIFPGSGSTYYNEKFKGKATLTVD
KSSSTVYMLLSSLTSEDSAVYFCAREAKLGRDYFDYWGQGTTLTVSS
SEQ ID NO: 56 5G7 VH Domain
QIQLVQSGPELKKPGETVKISCKASGYTFTTYGMSWVKQAPGKGLKWMGWINTYSGVPTYADDFKGRFAFSLE
TSASTAYLQINNLKNEDTTTYFCARFPYDFDGYFDVWGTGTAVTVSS
SEQ ID NO: 57 5H1 VH Domain
EFQLQQSGPELVKPGASVKISCKASGYSFTDYNMNWVKQSNGKSLAWIGVINPNYGTTNYNQKFKGKATLTVD
QSSSTAYMQLNSLTSEDSAVYYCARNYYGSTYDGYFDYWGQGTTLTVSS
SEQ ID NO: 58 6B2 VH Domain
QVQLQQSGAELMKPGASVKISCKATGYTINGYWIEWVKERPGHGLEWIGEILPGSGSTNYNEKFKGKATFTAD
TSSNTAYMQLSSLTTEDSAIYYCARGMEGAMDYWGQGTSVTVSS
SEQ ID NO: 59 6B3 VH Domain
QVQLQQPGAELVMPGASVRLSCKASGYTFTSYWMHWVKQRPGQGLEWIGEIDPSESYPNYNQNFKGKATLTVD
KSSSTAYMQLSSLTSEDSAVYYCARSYYGRSGYAMDYWGQGTSVTVSS
SEQ ID NO: 60 600000 VH Domain
QVQLTESGPGLVAPSQSLSITCTVSGFSLTNYIISWVRQPPGKGLEWLGVIWTGGGTNYNSALKSRLSISKDD
SKSQVFLKMNSLQTDDTARYYCARNEAVVAIFDWYFDVWGTGTTVTVSS
SEQ ID NO: 61 6F3 VH Domain
EFQLQQSGPELVKPGASVKISCKASGYSFTDYNMNWVKQSNGKSLEWIGVINPNYGTTSYNQKFKGKATLTVD
QSSSTAYMQLNSLTSEDSAVYYCARNYYGNNYDGYFDYWGQGTTLTVSS
SEQ ID NO: 62 6F4 VH Domain
QIQLVQSGPELKKPGETVKISCKASGYTFTMYGMSWVKQAPGKGLKWMGWINTYSGVPTYADDFKGRFAFSLE
TSANTAYLQINNLKNEDTATYFCARFPYDYDGYFDVWGTGTTVTVSS
SEQ ID NO: 63 9B3 VH Domain
EVQLQQSGPELVKPGASVKISCKASGYTFTDYYMNWVKQSHGKSLEWIGDINPNNGGTTYNQKFKGKATLTVD
KSSSTAYMELRSLTSEDSAVYYCARRYYSSGYDGYFDVWGTGTTVTVSS
SEQ ID NO: 64 15H3 VH Domain
EVQLQQSGPELVKPGASVKMSCKASGSTFTSYVMHWVKQKPGQGLEWIGYSNPYNDGTKYNEKFKGKATLTSD
KSSSTAYMELSSLTSEDSAVYYCARLNVLYYFDNWGQGTTLTVSS
SEQ ID NO: 65 8C3 VH Domain
QVQLQQPGTELVKPGASVKLSCKASGYTFTSYWMHWVKQRPGQGLEWIGNINPGNGGTDYNEKIKSKATLTVD
KSTSTAYMQLSSLTSEDSAVYYCARGGGYYGYDGYWYFDVWGTGTTVTVSS
SEQ ID NO: 66 4C7 VH Domain
EVQLQQSGPVLVKPGPSVKISCEASGFTFTDYYMHWVKQNHGKSLEWIGLVYPYNGDTIYNQKFKGKATLTVD
TSSSTAYMDLHSLTSEDSAVYYCARGANWGDYWGQGTTLTVSS
SEQ ID NO: 67 4D4 VH Domain
QVQLQQPGTELVKPGASVKLSCKASGYTFTSYWMHWVKQRPGQGLEWIGNISPSNGGTNYNENFKSKATLTVD
KSSSTAYMQLSSLTSEDSAVYYCATYYVDYWGQGTTLTVSS
SEQ ID NO: 68 6A3 VH Domain
QVTLKESGPGILQSSQTLSLTCSFSGFSLSTSGMGVSWIRQPSGKGLEWLAHIYWDDDKRYNPSLKSRLTISK
DTSRNQVFLKITSVDTADTATYYCARRVYGYDPYAMNYWGPGTSVTVSS
SEQ ID NO: 69 8B8 VH Domain
EVQLQQSGPELVKPGASVKISCKASGYTFTDYYMNWVKQSHGKSLEWIGDINPNNGGTSYNQKFKGKATLTVD
KSSSTAYMELRSLTSEDSAVYYCAPHYYGSSYDWYFDVWGTGTTVTVSS
SEQ ID NO: 70 9B1 VH Domain
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCAREDDSRYWYFDVWGTGTTVTVSS
SEQ ID NO: 71 9B2 VH Domain
QIQFVQSGPELKKPGETVKISCKASVYTFTEYPMHWVKQAPGKGFKWMGWINTYSGEPTYADDFKGRFAFSLE
TSASTAYLQINNLKNEDTASYFCAREGWLDAMDYWGQGTSVTVSS
SEQ ID NO: 72 9C1 VH Domain
QVQLKQSGPGLVQPSQSLSITCTVSGFSLTTFGVHWVRQSPGKGLEWLGVIWSGGSTDYNAAFISRLSISKDN
SKSQVFFKMNSLQVDDTAIYYCAPRLGLRAYWGQGTLVTVSA
SEQ ID NO: 73 9G1 VH Domain
QVQLKQSGPGLVQPSQSLSITCTVSGFSLTSYGVHWVRQSPGKGLEWLGVIWSGGTTDYNAAFISRLSISKDN
SKSQVFFKMNSLQADDTAIYYCARMGGTGYFDVWGTGTTVTVSS
SEQ ID NO: 74 3G6 VH Domain
QVQLKQSGPGLVQPSQSLSITCTVSGFSLTSYGVHWVRQSPGKGLEWLGVIWSGGTTDYNAAFISRLSISKDN
SKSQVFFKMNSLQADDTAIYYCARMGGTGYFDVWGTGTTVTVSS
SEQ ID NO: 75 3.9 VH-VL VH Domain
QVQLVQSGAEVKKPGASVKVSCKASGYTFTDYNMHWVRQAPGQGLEWMGYIYPYNGGTAYNQKFKNRVTMTRD
TSTSTVYMELSSLRSEDTAVYYCARGLDVMDYWGQGTLVTVSS
SEQ ID NO: 76 3.9 VL-VH VH Domain
QVQLVQSGAEVKKPGASVKVSCKVSGYTFTDYNMHWVRQAPGKGLEWMGYIYPYNGGTAYNQKFKNRVTMTED
TSTDTAYMELSSLRSEDTAVYYCARGLDVMDYWGQGTLVTVSS
SEQ ID NO: 77 2A07 VL Domain
SYELMQPPSVSVSPGQTASITCSGDKLGDKYVSWYQQKPGQSPVLVIYQDTKRPSGIPERFSGSNSGNTATLT
ISGTQAMDEADYYCQAWDSGTAIFGGGTKVTVL
SEQ ID NO: 78 2G08 VL Domain
QAGLTQPPSVSVSPGQTASITCSGDKLGDKYVSWYQQKPGQSPVLVIYQDSKRPSGIPERFSGSNSGNTATLT
ISGTQAMDEADYYCQAWDSSTVVFGGGTKVTVL
SEQ ID NO: 79 3000000000 VL Domain
QAGLTQPPSVSVSPGQTASITCFGDKLGHKYVSWYQQKPGQSPVLVIYQDSKRPSGIPERFSGSNSGNTATLT
ISGTQAMDEADYYCQAWDSSTVVFGGGTKLTVL
SEQ ID NO: 80 ′2E03 VL Domain
QPVLTQPPSVSVSPGQTASITCSGDKLGDKFTSWYQQRPGQSPVLVIYQDNKRPSGIPERFSGSNSGNTATLT
ISRVEAGDEADYYCQVWDSSSDHWVFGGGTQLTVL
SEQ ID NO: 81 2A05 VL Domain
QSVLTQPPSVSVSPGQTARISCSGDKLGDKYVSWYQQKPGQSPVLVIYEDSKRPSGIPERLSGSNSGNTATLT
ISGTQAMDEADYYCQAWDSSTVVFGGGTKLTVL
SEQ ID NO: 82 1A01 VL Domain
LSELTQDPAVSVALGQTVRITCQGDSLRNYYASWYQQKPGQAPVLVIYGKNNRPSGIPDRFSGSRSGNTASLT
ITGAQAEDEADYYCNSRDSSGNHPVVFGGGTKLTVL
SEQ ID NO: 83 1A11 VL Domain
LSELTQDPAVSVALGQTVRITCQGDSLRSYYASWYQQKPGQAPVLVIYGKNNRPSGIPDRFSGSSSGNTASLT
ITGAQAEDEADYYCNSRDSSGNHLVFGGGTKLTVL
SEQ ID NO: 84 1000000000 VL Domain
LSELTQDPAVSVALGQTVRITCQGDSLRSYYASWYQQKPGQAPVLVIYGKNNRPSGIPDRFSGSSSGNTASLT
ITGAQAEDEADYYCNSRDSSGNHVIFGGGTKLTVL
SEQ ID NO: 85 3B04 VL Domain
LPVLTQPPSVSVSPGQTASITCSGDKLGDKYASWYQQKPGQSPVLIIYQDTKRASGIPERFSGSNSGNTATLT
ISGTQAVDEADYYCQAFDSSAAHFVFGAGTKLTVL
SEQ ID NO: 86 200000 VL Domain
SYELMQPPSVSVSPGQTASITCSGDKLGDKYASWYQQKPGQSPVLVIYQDNKRPSGIPERFSGSNSGNTATLT
ISGTQAMDEADYYCQTWDSSTAVFGGGTKVTVL
SEQ ID NO: 87 2B2 VL Domain
DIQMTQSPASLSASVGETVTITCRASENIYSFLAWYQQKQGKSPQLLVYNAKTLAEGVPSRFSGSGSGTQFSL
KINSLOPEDFGSYYCQHHYGIPPTFGGGTKLEIK
SEQ ID NO: 88 3D4-LC1 VL Domain
QIVLTQSPAIMSASPGEKVTMTCSASSSVSYMYWYLQKPGSSPRLLIYDTSNLASGVPVRFSGSGSGTSYSLT
ISRMEAEDAATYYCQQWSSYPLTFGAGTKLELK
SEQ ID NO: 89 3D4-LC2 VL Domain
DVLMTQTPLSLPVSLGDQASISCRSSQSIVHSNGNTYLEWYLQKPGQSPKLLIYKVSNRFSGVPDRFSGSGSG
TDFTLKISRVEAEDLGLYYCFQGSHVPYTFGGGTKLEIK
SEQ ID NO: 90 4A5 VL Domain
DIVLTQSPASLAVSLGQRATISCRASESVDNYGISFMHWYQQKPGQPPKFLIYRASNLESGIPARFSGSGSRT
DFTLTINPVETDDVATYYCQQSNKDPRTFGGGTKLEIK
SEQ ID NO: 91 400000000 VL Domain
DIVMTQSPATLSVTPGDRVSLSCRASQSISDYLHWYQQKSHESPRLLIKYASQSISGIPSRFSGSGSGSDFTL
SINSVEPEDVGVYYCQNGHSFPLTFGAGTKLELK
SEQ ID NO: 92 4G6 VL Domain
DIVLTQSPASLAVSLGQRATISCRASESVSIHASHLLHWYQQKPGQPPKLLIYAASNLESGVPARFSGSGSET
DFTLNIHPVEEEDAATYFCQQSIEDPWTFGGGTKLEIK
SEQ ID NO: 93 500 VL Domain
DIVMTQSHKFMSTSVGDRVSITCKASQDVSTAVAWCQQKPGQSPKLLIYSASYRYTGVPDRFTGSGSGTDFTF
TISSVQAEDLAVYYCQQHYSTPYTFGGGTRLEIK
SEQ ID NO: 94 5G7 VL Domain
DIVMTQSHKFMSTSVGDRVSITCKASQDVGTAVAWYQEKPGQSPKILIYWASTRHTGVPDRFTGSGSGTDFTL
TISNVQSEDLADYFCQQYSSYPLTFGAGTKLELK
SEQ ID NO: 95 5H1 VL Domain
DIVLTQSPASLAVSLGQRATISCRASESVDNYGISFMHWYQQKPGQPPKFLIYRASNLESGIPARFSGSGSRT
DFTLTINPVETDDVATYYCQQSNKDPRTFGGGTKLEIT
SEQ ID NO: 96 6B2 VL Domain
DIVMTQSHKFMSTSVGDRVSITCKASQDVSTAVAWYQKKPGQSPKLLIYSASYRYTGVPDRFTGSGSGTDFTF
TISSVQAEDLAVYYCQQHYSTPPTFGGGTKLEIK
SEQ ID NO: 97 6B3 VL Domain
NIVMTQSPKSTSMSVGERVTLNCKASENVGTYVSWYQQKPEQSPKLLIYGASNRYTGVPDRFTGSGSATDFTL
TISSVQAEDLADYHCGQSYSYPPFTFGSGTKLEIK
SEQ ID NO: 98 600000 VL Domain
DIQMTQTTSSLSASLGDRVTISCRASQDISNYLNWYQQKPDGAVKLLIYYTSRLHSGVPSRFSGSGSGTDFSL
TISNLEQEDFATYFCQQGNTLPWTFGGGTKLEIK
SEQ ID NO: 99 6F3 VL Domain
DIVLTQSPASLAVSLGQRATISCRASESVDNYGISFMHWYQQKPGQPPKFLIYRASNLESGIPARFSGSGSRT
DFTLTINPVETDDVATYYCQQSNKDPRTFGGGTKLEIT
SEQ ID NO: 100 6F4 VL Domain
DIVMTQSHKFMSTSVGDRVSITCKASQDVGTAVAWYQQKPGQSPKLLIYWASTRHTGVPDRFTGSGSGTDFTL
AISNVQSEDLADYFCHQFSSYPLTFGAGTRLELK
SEQ ID NO: 101 9B3 VL Domain
DIVLTQSPASLAVSLGQRATISCRASENVDNYGISFMHWYQQKPGQPPKFLIYRASNLEYGIPARFSGSGSRT
DFTLTINPVETDDVATYYCQQSNKDPLTFGAGTKLELK
SEQ ID NO: 102 15H3 VL Domain
DIVLTQSPASLAVSLGQRATISCRASKSVSTSGYTYMHWYQQKPGQPPKLLIYLASNLESGVPARFSGSGSGT
DFTLNIHPVEEEDAATYYCQHSRELPLTFGAGTKLELK
SEQ ID NO: 103 8C3 VL Domain
DIVMTQAAFSIPVTLGTSTSISCRSTKSLLHSNGITYLYWYLQKPGQSPQLLIYQMSNLASGVPDRFSSSGSG
TDFTLRISRVEAEDVGVYYCAQNLELPWTFGGGTKLEIK
SEQ ID NO: 104 4C7 VL Domain
DVVMTQTPLSLPVSLGDQASISCRSSQSLVHSNGNTYLHWFLQKPGQSPKLLIYKVSNRFSGVPDRFSGSGSG
TDFTLKISRVEAEDLGLYFCSQSTHVPPTFGGGTKLEIK
SEQ ID NO: 105 4D4 VL Domain
DVVMTQTPLTLSVTIGQPASISCKSTQSLLDSDGKTYLNWFLQRPGQSPKRLIYLVSKLDSGVPDRFTGSGSG
TDFTLKISRVEAEDLGLYYCWQGTHFPQTFGGGTKLEIK
SEQ ID NO: 106 6A3 VL Domain
QIVLTQSPALMSASPGEKVTMTCSASSSVSYMYWYQQKPRSSPKPWIYLTSTLASGVPARFSGSGSGTSYSLT
ISSMEAEDAATYYCQQWSSNPYTFGGGTKLEIK
SEQ ID NO: 107 8B8 VL Domain
DIVLTQSPASLAVSLGQRATISCRASESVDNYGISFMHWYQQKPGQPPKLLIYRASNLESGIPARFSGSGSRT
DFTLTINPVETDDVATYYCQQSNKDPLTFGAGTKLELK
SEQ ID NO: 108 9B1 VL Domain
DIVMTQSHKFMSTSVGDRVSITCKASQDVGTAVAWYQQKPGQSPKLLIYWASTRHTGVPDRFTGSGSGTDFTL
TISNVQSEDLADYFCQQYSSYPYTFGGGTKLEIK
SEQ ID NO: 109 9B2 VL Domain
DIVMTQSQKFMSTIVGDRVSITCKASQNVGTAVAWYQQKPGQSPKLLIYSASNRYTGVPDRFTGSGSGTDFTL
TISNMQSEDLADYFCQQYSSYTWTFGGGTKLEIK
SEQ ID NO: 110 9C1 VL Domain
DIKMTQSPSSMYASLGERVTITCKASQDINSYLSWFQQKPGKSPKTLIYRANRLVDGVPSRFSGSGSGQDYSL
TISSLEYEDMGIYYCLQYDEFPYTFGGGTKLEVK
SEQ ID NO: 111 9G1 VL Domain
NIVMTQSPKSMSMSVGERVTLSCKAGENVGPYVSWYQQKPEQSPKLLIYGASNRFTGVPDRFTGSGSATDFTL
TISSVQAEDLADYHCGQSYSYPFTFGSGTKLEIK
SEQ ID NO: 112 3G6 VL Domain
DIVMTQAAFSNPVTLGTSASISCRSSKSLLHSNGITYLYWYLQKPGLSPQLLIYHMSNLASGVPDRFSSSGSG
TDFTLRISRVEAEDVGVYYCAQNLELPWTFGGGTKLEIK
SEQ ID NO: 113 3.9 VH-VL VL Domain
AIQLTQSPSSLSASVGDRVTITCRASENIYSYLAWYQQKPGKAPKLLIYNAKILAEGVPSRFSGSGSGTDFTL
TISSLQPEDFATYYCQHHYVSPRTFGQGTKLEIK
SEQ ID NO: 114 3.9VL-VH VL Domain
DIQMTQSPSSLSASVGDRVTITCRASENIYSYLAWYQQKPGKAPKLLIYNAKILAEGVPSRFSGSGSGTDFTL
TISSLQPEDFATYYCQHHYVSPRTFGQGTKLEIK
SEQ ID NO: 115 CDR H1 #1

GYSFTSYWIG

SEQ ID NO: 116 CDR H1 #2

GFTFSSYGMH

SEQ ID NO: 117 CDR H1 #3

GYTFTNFWMH

SEQ ID NO: 118 CDR H1 #4

GYTFTSYTMH

SEQ ID NO: 119 CDR H1 #5

GYSFTDYNMN

SEQ ID NO: 120 CDR H1 #6

GYTFTDYYID

SEQ ID NO: 121 CDR H1 #7

GYTFTDYYMN

SEQ ID NO: 122 CDR H1 #8

GYTFTDYYIN

SEQ ID NO: 123 CDR H1 #9

GYTFTTYGMSWV

SEQ ID NO: 124 CDR H1 #10

GYTINGY

SEQ ID NO: 125 CDR H1 #11

GYTFTSYWMH

SEQ ID NO: 126 CDR H1 #12

GFSLTNYIIS

SEQ ID NO: 127 CDR H1 #13

GYTFTMYGMSWV

SEQ ID NO: 128 CDR H1 #14

GSTFTSYVMH

SEQ ID NO: 129 CDR H1 #15

GFTFTDYYMH

SEQ ID NO: 130 CDR H1 #16

GFSLSTSGMGVS

SEQ ID NO: 131 CDR H1 #17

GYTFTDYNMD

SEQ ID NO: 132 CDR H1 #18

VYTFTEYPMHWV

SEQ ID NO: 133 CDR H1 #19

GFSLTTFGVH

SEQ ID NO: 134 CDR H1 #20

GFSLTSYGVH

SEQ ID NO: 135 CDR H1 #21

GYTFTDYNMH

SEQ ID NO: 136 CDR H2 #1

IIYPGDSDTRYSPSFQG

SEQ ID NO: 137 CDR H2 #2

FIRYDGSNKYYADSVKG

SEQ ID NO: 138 CDR H2 #3

INPSSDYTKYNQKFKG

SEQ ID NO: 139 CDR H2 #4

INPSSGYTKYNQKFKD

SEQ ID NO: 140 CDR H2 #5

INPNYGTTSYNQKFKG

SEQ ID NO: 141 CDR H2 #6

IFPGTNSTYYNEKFKG

SEQ ID NO: 142 CDR H2 #7

INPYSGGTSYNQKFKG

SEQ ID NO: 143 CDR H2 #8

IFPGSGSTYYNEKFKG

SEQ ID NO: 144 CDR H2 #9

WINTYSGVPTYADDFK

SEQ ID NO: 145 CDR H2 #10

INPNYGTTNYNQKFKG

SEQ ID NO: 146 CDR H2 #11

ILPGSGSTNYNEKFKG

SEQ ID NO: 147 CDR H2 #12

IDPSESYPNYNQNFKG

SEQ ID NO: 148 CDR H2 #13

IWTGGGTNYNSALKS

SEQ ID NO: 149 CDR H2 #14

INPNNGGTTYNQKFKG

SEQ ID NO: 150 CDR H2 #15

SNPYNDGTKYNEKFKG

SEQ ID NO: 151 CDR H2 #16

INPGNGGTDYNEKIKS

SEQ ID NO: 152 CDR H2 #17

VYPYNGDTIYNQKFKG

SEQ ID NO: 153 CDR H2 #18

ISPSNGGTNYNENFKS

SEQ ID NO: 154 CDR H2 #19

IYWDDDKRYNPSLKS

SEQ ID NO: 155 CDR H2 #20

INPNNGGTSYNQKFKG

SEQ ID NO: 156 CDR H2 #21

INPNNGGTIYNQKFKG

SEQ ID NO: 157 CDR H2 #22

WINTYSGEPTYADDFK

SEQ ID NO: 158 CDR H2 #23

IWSGGSTDYNAAFIS

SEQ ID NO: 159 CDR H2 #24

IWSGGTTDYNAAFIS

SEQ ID NO: 160 CDR H2 #25

YIYPYNGGTAYNQKFKN

SEQ ID NO: 161 CDR H3 #1

ARYIQGLGYYFDY

SEQ ID NO: 162 CDR H3 #2

AKPSRGYSRSLDY

SEQ ID NO: 163 CDR H3 #3

ARLQSGWLHAFDI

SEQ ID NO: 164 CDR H3 #4

ARLKWSGLSHYYYYYMDV

SEQ ID NO: 165 CDR H3 #5

ARLPLGLQVGFDY

SEQ ID NO: 166 CDR H3 #6

ARVRYSYDLNFDY

SEQ ID NO: 167 CDR H3 #7

ARDDYSDFGFAY

SEQ ID NO: 168 CDR H3 #8

AREANWDDVDY

SEQ ID NO: 169 CDR H3 #9

ARNYYSSSYDGYFDY

SEQ ID NO: 170 CDR H3 #10

ARSGLRDFDY

SEQ ID NO: 171 CDR H3 #11

ASVSSYGNYFDY

SEQ ID NO: 172 CDR H3 #12

AREAKLGRDYFDY

SEQ ID NO: 173 CDR H3 #13

ARFPYDFDGYFDV

SEQ ID NO: 174 CDR H3 #14

ARNYYGSTYDGYFDY

SEQ ID NO: 175 CDR H3 #15

ARGMEGAMDY

SEQ ID NO: 176 CDR H3 #16

ARSYYGRSGYAMDY

SEQ ID NO: 177 CDR H3 #17

ARNEAVVAIFDWYFDV

SEQ ID NO: 178 CDR H3 #18

ARNYYGNNYDGYFDY

SEQ ID NO: 179 CDR H3 #19

ARFPYDYDGYFDV

SEQ ID NO: 180 CDR H3 #20

ARRYYSSGYDGYFDV

SEQ ID NO: 181 CDR H3 #21

ARLNVLYYFDN

SEQ ID NO: 182 CDR H3 #22

ARGGGYYGYDGYWYFDV

SEQ ID NO: 183 CDR H3 #23

ARGANWGDY

SEQ ID NO: 184 CDR H3 #24

ATYYVDY

SEQ ID NO: 185 CDR H3 #25

ARRVYGYDPYAMNY

SEQ ID NO: 186 CDR H3 #26

APHYYGSSYDWYFDV

SEQ ID NO: 187 CDR H3 #27

AREDDSRYWYFDV

SEQ ID NO: 188 CDR H3 #28

AREGWLDAMDY

SEQ ID NO: 189 CDR H3 #29

APRLGLRAY

SEQ ID NO: 190 CDR H3 #30

ARMGGTGYFDV

SEQ ID NO: 191 CDR H3 #31

ARGLDVMDY

SEQ ID NO: 192 CDR L1 #1

SGDKLGDKYVS

SEQ ID NO: 193 CDR L1 #2

FGDKLGHKYVS

SEQ ID NO: 194 CDR L1 #3

SGDKLGDKFTS

SEQ ID NO: 195 CDR L1 #4

QGDSLRNYYAS

SEQ ID NO: 196 CDR L1 #5

QGDSLRSYYAS

SEQ ID NO: 197 CDR L1 #6

SGDKLGDKYAS

SEQ ID NO: 198 CDR L1 #7

NIYSFLAWY

SEQ ID NO: 199 CDR L1 #8

SVSYMYWY

SEQ ID NO: 200 CDR L1 #9

SIVHSNGNTYLEWY

SEQ ID NO: 201 CDR L1 #10

SVDNYGISFMHWY

SEQ ID NO: 202 CDR L1 #11

SISDYLHWY

SEQ ID NO: 203 CDR L1 #12

SVSIHASHLLHWYQ

SEQ ID NO: 204 CDR L1 #13

DVSTAVAWC

SEQ ID NO: 205 CDR L1 #14

DVGTAVAWY

SEQ ID NO: 206 CDR L1 #15

DVSTAVAWY

SEQ ID NO: 207 CDR L1 #16

NVGTYVSWY

SEQ ID NO: 208 CDR L1 #17

DISNYLNWY

SEQ ID NO: 209 CDR L1 #18

NVDNYGISFMHWY

SEQ ID NO: 210 CDR L1 #19

SVSTSGYTYMHWY

SEQ ID NO: 211 CDR L1 #20

SLLHSNGITYLYWY

SEQ ID NO: 212 CDR L1 #21

SLVHSNGNTYLHWF

SEQ ID NO: 213 CDR L1 #22

SLLDSDGKTYLNWF

SEQ ID NO: 214 CDR L1 #23

NVGTAVAWY

SEQ ID NO: 215 CDR L1 #24

DINSYLSWF

SEQ ID NO: 216 CDR L1 #25

NVGPYVSWY

SEQ ID NO: 217 CDR L1 #26

RASENIYSYLA

SEQ ID NO: 218 CDR L2 #1

QDTKRPS

SEQ ID NO: 219 CDR L2 #2

QDSKRPS

SEQ ID NO: 220 CDR L2 #3

QDNKRPS

SEQ ID NO: 221 CDR L2 #4

EDSKRPS

SEQ ID NO: 222 CDR L2 #5

GKNNRPS

SEQ ID NO: 223 CDR L2 #6

QDTKRAS

SEQ ID NO: 224 CDR L2 #7

KTLAEGVPS

SEQ ID NO: 225 CDR L2 #8

NLASGVPV

SEQ ID NO: 226 CDR L2 #9

SNRFSGVPD

SEQ ID NO: 227 CDR L2 #10

NLESGIPA

SEQ ID NO: 228 CDR L2 #11

QSISGIPS

SEQ ID NO: 229 CDR L2 #12

NLESGVPA

SEQ ID NO: 230 CDR L2 #13

YRYTGVPD

SEQ ID NO: 231 CDR L2 #14

TRHTGVPD

SEQ ID NO: 232 CDR L2 #15

NRYTGVPD

SEQ ID NO: 233 CDR L2 #16

SRLHSGVPS

SEQ ID NO: 234 CDR L2 #17

NLEYGIPA

SEQ ID NO: 235 CDR L2 #18

NLASGVPD

SEQ ID NO: 236 CDR L2 #19

SKLDSGVPD

SEQ ID NO: 237 CDR L2 #20

TLASGVPA

SEQ ID NO: 238 CDR L2 #21

NRLVDGVPS

SEQ ID NO: 239 CDR L2 #22

NRFTGVPD

SEQ ID NO: 240 CDR L2 #23

NAKILAE

SEQ ID NO: 241 CDR L3 #1

QAWDSGTAI

SEQ ID NO: 242 CDR L3 #2

QAWDSSTVV

SEQ ID NO: 243 CDR L3 #3

QVWDSSSDHWV

SEQ ID NO: 244 CDR L3 #4

NSRDSSGNHPVV

SEQ ID NO: 245 CDR L3 #5

NSRDSSGNHLV

SEQ ID NO: 246 CDR L3 #6

NSRDSSGNHVI

SEQ ID NO: 247 CDR L3 #7

QAFDSSAAHFV

SEQ ID NO: 248 CDR L3 #8

QTWDSSTAV

SEQ ID NO: 249 CDR L3 #9

QHHYGIPPT

SEQ ID NO: 250 CDR L3 #10

QQWSSYPLT

SEQ ID NO: 251 CDR L3 #11

FQGSHVPYT

SEQ ID NO: 252 CDR L3 #12

QQSNKDPRT

SEQ ID NO: 253 CDR L3 #13

QNGHSFPLT

SEQ ID NO: 254 CDR L3 #14

QQSIEDPWT

SEQ ID NO: 255 CDR L3 #15

QHYSTPYT

SEQ ID NO: 256 CDR L3 #16

QQYSSYPLT

SEQ ID NO: 257 CDR L3 #17

QQHYSTPPT

SEQ ID NO: 258 CDR L3 #18

GQSYSYPPFT

SEQ ID NO: 259 CDR L3 #19

QQGNTLPWT

SEQ ID NO: 260 CDR L3 #20

HQFSSYPLT

SEQ ID NO: 261 CDR L3 #21

QQSNKDPLT

SEQ ID NO: 262 CDR L3 #22

QHSRELPLT

SEQ ID NO: 263 CDR L3 #23

AQNLELPWT

SEQ ID NO: 264 CDR L3 #24

SQSTHVPPT

SEQ ID NO: 265 CDR L3 #25

WQGTHFPQT

SEQ ID NO: 266 CDR L3 #26

QQWSSNPYT

SEQ ID NO: 267 CDR L3 #27

QQYSSYPYT

SEQ ID NO: 268 CDR L3 #28

QQYSSYTWT

SEQ ID NO: 269 CDR L3 #29

LQYDEFPYT

SEQ ID NO: 270 CDR L3 #30

GQSYSYPFT

SEQ ID NO: 271 CDR L3 #31

QHHYVSPRT

SEQ ID NO: 272 Linker(G4S)3

GGGGSGGGGSGGGGS

SEQ ID NO: 273 CD28 Co-stimulatory domain
IEVMYPPPYLDNEKSNGTIIHVKGKHLCPSPLFPGPSKPFWVLVVVGGVLACYSLLVTVAFIIFWVRSKRSRL
LHSDYMNMTPRRPGPTRKHYQPYAPPRDFAAY
SEQ ID NO: 274 CD3 zeta Signaling Domain
MKWKALFTAAILQAQLPITEAQSFGLLDPKLCYLLDGILFIYGVILTALFLRVKFSRSADAPAYQQGQNQLYN
ELNLGRREEYDVLDKRRGRDPEMGGKPQRRKNPQEGLYNELQKDKMAEAYSEIGMKGERRRGKGHDGLYQGLS
TATKDTYDALHMQALPPR
SEQ ID NO: 275 Human CD8α transmembrane domain

IYIWAPLAGTCGVLLLSLVIT

SEQ ID NO: 276 Human CD8α transmembrane domain

IYIWAPLAGTCGVLLLSLVITLYCNHRN

SEQ ID NO: 277 Human CD28 transmembrane domain

FWVLVVVGGVLACYSLLVTVAFIIFWV

SEQ ID NO: 278 OX40 Co-stimulatory Domain

ALYLLRRDQRLPPDAHKPPGGGSFRTPIQEEQADAHSTLAKI

SEQ ID NO: 279: 4-1BB Co-stimulatory Domain

KRGRKKLLYIFKQPFMRPVQTTQEEDGCSCRFPEEEEGGCEL

SEQ ID NO: 280 T2A cleavage sequence

GSGEGRGSLLTCGDVEENPGP

SEQ ID NO: 281 P2A cleavage sequence

GSGATNFSLLKQAGDVEENPGP

SEQ ID NO: 282 E2A cleavage sequence

GSGQCTNYALLKLAGDVESNPGP

SEQ ID NO: 283 F2A cleavage sequence

GSGVKQTLNFDLLKLAGDVESNPGP

SEQ ID NO: 284 Tbx21 Intracellular Signaling Domain
MGIVEPGCGDMLTGTEPMPGSDEGRAPGADPQHRYFYPEPGAQDADERRGGGSLGSPYPGGALVPAPPSRFLG
AYAYPPRPQAAGFPGAGESFPPPADAEGYOPGEGYAAPDPRAGLYPGPREDYALPAGLEVSGKLRVALNNHLL
WSKENQHQTEMIITKOGRRMFPFLSFTVAGLEPTSHYRMFVDVVLVDQHHWRYQSGKWVQCGKAEGSMPGNRL
YVHPDSPNTGAHWMROEVSFGKLKLTNNKGASNNVTQMIVLOSLHKYQPRLHIVEVNDGEPEAACNASNTHIF
TFOETQFIAVTAYONAEITOLKIDNNPFAKGFRENFESMYTSVDTSIPSPPGPNCQFLGGDHYSPLLPNQYPV
PSRFYPDLPGQAKDVVPQAYWLGAPRDHSYEAEFRAVSMKPAFLPSAPGPTMSYYRGQEVLAPGAGWPVAPQY
PPKMGPASWFRPMRTLPMEPGPGGSEGRGPEDQGPPLVWTEIAPIRPESSDSGLGEGDSKRRRVSPYPSSGDS
SSPAGAPSPFDKEAEGQFYNYFPN
SEQ ID NO: 285 E2S-VP64 Intracellular Signaling Domain
MAQAALEPGEKPYACPECGKSFSTSGSLVRHQRTHTGEKPYKCPECGKSFSRNDALTEHQRTHTGEKPYKCPE
CGKSFSSKKHLAEHQRTHTGEKPYACPECGKSFSTSGELVRHQRTHTGEKPYKCPECGKSFSRSDKLVRHQRT
HTGEKPYKCPECGKSFSRSDHLTEHQRTHTGKKTSGQAGQASPKKKRKVGRADALDDFDLDMLGSDALDDFDL
DMLGSDALDDFDLDMLGSDALDDFDLDMLINYPYDVPDYAS
SEQ ID NO: 286 GAL4-VP64 Intracellular Signaling Domain
MKLLSSIEQACDICRLKKLKCSKEKPKCAKCLKNNWECRYSPKTKRSPLTRAHLTEVESRLERLEOLFLLIFP
REDLDMILKMDSLQDIKALLTGLFVQDNVNKDAVTDRLASVETDMPLTLROHRISATSSSEESSNKGQRQLTV
SAAAGGSGGSGGSDALDDFDLDMLGSDALDDFDLDMLGSDALDDFDLDMLGSDALDDFDLDMLGS
*SEQ ID NOs: 39-114 show both underlined and bolded amino acids. The
underlined amino acids represent CDRs 1-3, respectively, in either the
Variable Heavy Chain Region (SEQ ID NOs: 39-76) or the Variable Light
Chain Region (SEQ IDs: 77-114) annotated by the IMGT method. The
bolded amino acids represent CDRs 1-3, respectively, in either the
Variable Heavy Chain Region (SEQ ID NOs: 39-76) or the Variable Light
Chain Region (SEQ ID NOs: 77-114) annotated by the Kabat method.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for modulating signaling in a cell, said method comprising transforming the cell with
a nucleic acid sequence encoding a chimeric receptor polypeptide, wherein the chimeric receptor polypeptide comprises an extracellular domain, a transmembrane domain, and an intracellular domain, wherein the extracellular domain comprises a first antigen binding domain capable of binding to a first antigen on a CD11c⁺Tbet⁺ B cell, and wherein the intracellular domain comprises a transcriptional control unit and a proteolytic site; and
a nucleic acid sequence encoding a chimeric antigen receptor polypeptide, wherein the chimeric antigen receptor comprises a second antigen binding domain capable of binding to a second antigen present on the CD11c⁺T-bet⁺ B cell, wherein the nucleic acid sequence encoding the chimeric antigen receptor polypeptide is operably linked to a transcriptional control element to which the transcriptional control unit can bind; and
contacting the cell with a CD11c+T-bet+ B cell expressing the first antigen on its surface, wherein the contacting induces cleavage at the proteolytic site, thereby releasing the intracellular domain; and
wherein the transcriptional control unit activates the transcriptional control element operably linked to the nucleic acid sequence encoding the chimeric antigen receptor polypeptide, thereby activating expression of the chimeric antigen receptor polypeptide.

2. The method of claim 1, wherein the chimeric receptor polypeptide is a chimeric NOTCH receptor polypeptide.

3. The method of claim 2, wherein the chimeric NOTCH receptor is a SYNNOTCH® receptor.

4. The method of claim 3, wherein the first antigen binding domain is an antibody or antigen binding fragment.

5. The method of claim 4, wherein the first antigen binding domain is an antigen binding fragment selected from the group consisting of a Fab, a F(ab′)₂ fragment, a scFv, a scab, a dAb, a single domain heavy chain antibody, and a single domain light chain antibody.

6. The method of claim 4, wherein the first antigen is a B cell receptor.

7. The method of claim 6, wherein the B cell receptor is selected from the group consisting of CD19, CD20, and CD45R.

8. The method of claim 7, wherein the first antigen binding domain binds CD19.

9. The method of claim 8, wherein the first antigen binding domain comprises an scFv comprising a sequence at least 90% identical to one of SEQ ID NOs: 1-10.

10. The method of claim 9, wherein the first antigen binding domain comprises one of the following:
(a) a heavy chain variable domain comprising SEQ ID NO: 39 and a light chain variable domain comprising SEQ ID NO: 77;
(b) a heavy chain variable domain comprising SEQ ID NO: 40 and a light chain variable domain comprising SEQ ID NO: 78;
(c) a heavy chain variable domain comprising SEQ ID NO: 41 and a light chain variable domain comprising SEQ ID NO: 79;
(d) a heavy chain variable domain comprising SEQ ID NO: 42 and a light chain variable domain comprising SEQ ID NO: 80;
(e) a heavy chain variable domain comprising SEQ ID NO: 43 and a light chain variable domain comprising SEQ ID NO: 81;
(f) a heavy chain variable domain comprising SEQ ID NO: 44 and a light chain variable domain comprising SEQ ID NO: 82;
(g) a heavy chain variable domain comprising SEQ ID NO: 45 and a light chain variable domain comprising SEQ ID NO: 83;
(h) a heavy chain variable domain comprising SEQ ID NO: 46 and a light chain variable domain comprising SEQ ID NO: 84;
(i) a heavy chain variable domain comprising SEQ ID NO: 47 and a light chain variable domain comprising SEQ ID NO: 85; or
(j) a heavy chain variable domain comprising SEQ ID NO: 48 and a light chain variable domain comprising SEQ ID NO: 86.

11. The method of claim 4, wherein the second antigen is a receptor present on CD11c⁺T-bet B⁺ cells.

12. The method of claim 1, wherein the transcriptional control unit comprises a transcriptional activator.

13. The method of claim 1, wherein the second antigen binding domain is an antibody or antigen binding fragment.

14. The method of claim 13, wherein the second antigen binding domain is an antigen binding fragment selected from the group consisting of a Fab, a F(ab′)₂ fragment, a scFv, a scab, a dAb, a single domain heavy chain antibody and a single domain light chain antibody.

15. The method of claim 13, wherein the second antigen is a receptor present on CD11c⁺T-bet B⁺ cells.

16. The method of claim 15, wherein the second antigen is CD11c.

17. The method of claim 16, wherein the first antigen binding domain comprises an scFv comprising a sequence at least 90% identical to SEQ ID NO: 37 or SEQ ID NO: 38.

18. The method of claim 17, wherein the first antigen binding domain comprises either
(a) a heavy chain variable domain comprising SEQ ID NO: 75 and a light chain variable domain comprising SEQ ID NO: 113; or
(b) a heavy chain variable domain comprising SEQ ID NO: 76 and a light chain variable domain comprising SEQ ID NO: 114.

19. The method of claim 1, wherein the proteolytic site is cleavable by a member of the ADAM family of proteases.

20. The method of claim 1, wherein the first antigen binding domain is targeted to a first epitope and the second antigen binding domain is targeted to a second epitope.

21. The method of claim 1, wherein the first epitope and the second epitope are on the same target.

22. The method of claim 1, wherein the first epitope and the second epitope are on different targets.

23. The method of claim 1, wherein the first antigen binding domain and the second antigen binding domain bind the same antigen and the same epitope.

24. The method of claim 1, wherein the nucleic acid sequences are in separate vectors.

25. The method of claim 1, wherein the nucleic acid sequences are included in the same vector.

26. A method of treating a mammal having a disease, the method comprising administering to the mammal a cell transformed as in any one of claims 1-25.

27. The method of claim 26, wherein the disease is an autoimmune disorder.

28. The method of claim 26, wherein the autoimmune disorder is selected from a group consisting of lupus, rheumatoid arthritis, multiple sclerosis, insulin dependent diabetes mellitus, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, and polyarteritis nodosa.

29. A chimeric receptor polypeptide comprising an extracellular domain, a transmembrane domain, and an intracellular domain, wherein the extracellular domain comprises an antigen binding domain that binds antigens present on CD11c⁺Tbet⁺B cells, wherein the intracellular domain comprises a transcriptional control unit and a proteolytic site, and wherein the transcriptional control unit comprises a domain capable of activating a transcriptional control element.

30. The chimeric receptor polypeptide of claim 29, wherein the chimeric receptor polypeptide is a chimeric NOTCH receptor polypeptide.

31. The chimeric receptor polypeptide of claim 30, wherein the chimeric NOTCH receptor is a SYNNOTCH® receptor.

32. The chimeric receptor polypeptide of claim 31, wherein the antigen binding domain is an antibody or antigen binding fragment.

33. The chimeric receptor polypeptide of claim 32, wherein the antibody or antigen binding fragment binds to a B cell receptor selected from the group consisting of CD19, CD20, and CD45R.

34. The chimeric receptor polypeptide of claim 33, wherein the antigen binding domain binds CD19.

35. The chimeric receptor polypeptide of claim 34, wherein the antigen binding domain comprises an scFv comprising a sequence at least 90% identical to one of SEQ ID NOs: 1-10.

36. The chimeric receptor polypeptide of claim 35, wherein the antigen binding domain comprises one of the following:
(a) a heavy chain variable domain comprising SEQ ID NO: 39 and a light chain variable domain comprising SEQ ID NO: 77;
(b) a heavy chain variable domain comprising SEQ ID NO: 40 and a light chain variable domain comprising SEQ ID NO: 78;
(c) a heavy chain variable domain comprising SEQ ID NO: 41 and a light chain variable domain comprising SEQ ID NO: 79;
(d) a heavy chain variable domain comprising SEQ ID NO: 42 and a light chain variable domain comprising SEQ ID NO: 80;
(e) a heavy chain variable domain comprising SEQ ID NO: 43 and a light chain variable domain comprising SEQ ID NO: 81;
(f) a heavy chain variable domain comprising SEQ ID NO: 44 and a light chain variable domain comprising SEQ ID NO: 82;
(g) a heavy chain variable domain comprising SEQ ID NO: 45 and a light chain variable domain comprising SEQ ID NO: 83;
(h) a heavy chain variable domain comprising SEQ ID NO: 46 and a light chain variable domain comprising SEQ ID NO: 84;
(i) a heavy chain variable domain comprising SEQ ID NO: 47 and a light chain variable domain comprising SEQ ID NO: 85; or
(j) a heavy chain variable domain comprising SEQ ID NO: 48 and a light chain variable domain comprising SEQ ID NO: 86.

37. The chimeric receptor polypeptide of claim 33, wherein the B cell receptor is a receptor present on CD11c+T-bet B+ cells.

38. A pharmaceutical composition comprising the chimeric receptor polypeptide of any one of claims 29-37.

39. An isolated nucleic acid encoding the chimeric receptor polypeptide of any one of claims 29-37.

40. A vector comprising the nucleic acid of claim 39.

41. A cell comprising the nucleic acid of claim 39 or the vector of claim 40.

42. A chimeric antigen receptor polypeptide comprising (i) a single-chain variable fragment (scFv) having binding specificity for a CD11c⁺T-Bet⁺ B cell antigen, (ii) a transmembrane domain, (iii) at least one co-stimulatory domain, and (iv) an activating domain.

43. The chimeric antigen receptor polypeptide of claim 42, wherein the scFv fragment has binding specificity for CD11c.

44. The chimeric antigen receptor polypeptide of claim 43, wherein the scFv fragment comprises a sequence at least 90% identical to SEQ ID NO: 37 or SEQ ID NO: 38.

45. The chimeric antigen receptor polypeptide of claim 44, wherein the scFv fragment comprises either:
(a) a heavy chain variable domain comprising SEQ ID NO: 75 and a light chain variable domain comprising SEQ ID NO: 113; or
(b) a heavy chain variable domain comprising SEQ ID NO: 76 and a light chain variable domain comprising SEQ ID NO: 114.

46. An isolated nucleic acid encoding the chimeric antigen receptor polypeptide of claim 42 or claim 43.

47. A vector comprising the nucleic acid of claim 46.

48. A cell comprising the nucleic acid of claim 46 or the vector of claim 47.

49. A vector comprising the nucleic acid of claim 39 and the nucleic acid of claim 46.

50. A cell comprising the nucleic acid of claim 39 and the nucleic acid of claim 46, or the vector of claim 49.

51. The cell of any one of claim 41, 48, or 50, wherein the cell is an immune cell, a neuron, an epithelial cell, an endothelial cell, or a stem cell.

52. The cell of claim 51, wherein said cell is an immune cell selected from the group consisting of a T cell, a B cell, a monocyte, a natural killer cell, a dendritic cell, a macrophage, a regulatory T cell, a helper T cell, and a cytotoxic T cell.

53. A method of producing a chimeric receptor polypeptide, the method comprising culturing the cell of claim 41 in a culture medium under conditions sufficient to result in expression of the chimeric receptor polypeptide.

54. The method of claim 53, comprising recovering the chimeric receptor polypeptide from the cell and/or cell culture medium.

55. A chimeric receptor polypeptide produced by the method of claim 53 or claim 54.

56. A method of producing a chimeric antigen receptor polypeptide, the method comprising culturing the cell of claim 48 in a culture medium under conditions sufficient to result in expression of the chimeric receptor polypeptide.

57. The method of claim 56, comprising recovering the chimeric antigen receptor polypeptide from the cell and/or cell culture medium.

58. A chimeric antigen receptor polypeptide produced by the method of claim 56 or claim 57.


SEQUENCE LISTING (286 sequences)

SEQ ID NO: 1 - protein, 241 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVSTGGGGSGGGGSGGGGSSYELMQPPSVSVSPGQTASITCSGDKLGDKYVSWYQQKPGQSPVLVIYQDTKRPSGIPERFSGSNSGNTATLTISGTQAMDEADYYCQAWDSGTAIFGGGTKVTVL

SEQ ID NO: 2 - protein, 241 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVSTGGGGSGGGGSGGGGSQAGLTQPPSVSVSPGQTASITCSGDKLGDKYVSWYQQKPGQSPVLVIYQDSKRPSGIPERFSGSNSGNTATLTISGTQAMDEADYYCQAWDSSTVVFGGGTKVTVL

SEQ ID NO: 3 - protein, 241 aa, Homo sapiens
QLQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVSTGGGGSGGGGSGGGGSQAGLTQPPSVSVSPGQTASITCFGDKLGHKYVSWYQQKPGQSPVLVIYQDSKRPSGIPERFSGSNSGNTATLTISGTQAMDEADYYCQAWDSSTVVFGGGTKLTVL

SEQ ID NO: 4 - protein, 243 aa, Homo sapiens
QMQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKPSRGYSRSLDYWGQGTLVTVSTGGGGSGGGGSGGGGSQPVLTQPPSVSVSPGQTASITCSGDKLGDKFTSWYQQRPGQSPVLVIYQDNKRPSGIPERFSGSNSGNTATLTISRVEAGDEADYYCQVWDSSSDHWVFGGGTQLTVL

SEQ ID NO: 5 - protein, 241 aa, Homo sapiens
QVQLVESGAEVKKPGESLKISCKGSGYSFTSYWIGWVRQMPGKGLEWMGIIYPGDSDTRYSPSFQGQVTISADKSISTAYLQWSSLKASDTAMYYCARLQSGWLHAFDIWGQGTMVTVSTGGGGSGGGGSGGGGSQSVLTQPPSVSVSPGQTARISCSGDKLGDKYVSWYQQKPGQSPVLVIYEDSKRPSGIPERLSGSNSGNTATLTISGTQAMDEADYYCQAWDSSTVVFGGGTKLTVL

SEQ ID NO: 6 - protein, 249 aa, Homo sapiens
QVQLLQSAAEVKKPGESLKISCKGSGYSFTSYWIGWVRQMPGKGLEWMGIIYPGDSDTRYSPSFQGQITISADKSISTAYLQWSSLKASDTAMYYCARLKWSGLSHYYYYYMDVWGKGTTVTVSTGGGGSGGGGSGGGGSLSELTQDPAVSVALGQTVRITCQGDSLRNYYASWYQQKPGQAPVLVIYGKNNRPSGIPDRFSGSRSGNTASLTITGAQAEDEADYYCNSRDSSGNHPVVFGGGTKLTVL

SEQ ID NO: 7 - protein, 248 aa, Homo sapiens
QVQLLQSAAEVKKPGESLKISCKGSGYSFTSYWIGWVRQMPGKGLEWMGIIYPGDSDTRYSPSFQGQVTISADKSISTAYLQWSSLKASDTAMYYCARLKWSGLSHYYYYYMDVWGKGTTVTVSTGGGGSGGGGSGGGGSLSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTKLTVL

SEQ ID NO: 8 - protein, 248 aa, Homo sapiens
QVQLLQSAAEVKKPGESLKISCKGSGYSFTSYWIGWVRQMPGKGLEWMGIIYPGDSDTRYSPSFQGQVTISADKSISTAYLQWSSLKASDTAMYYCARLKWSGLSHYYYYYMDVWGKGTTVTVSTGGGGSGGGGSGGGGSLSELTQDPAVSVALGQTVRITCQGDSLRSYYASWYQQKPGQAPVLVIYGKNNRPSGIPDRFSGSSSGNTASLTITGAQAEDEADYYCNSRDSSGNHVIFGGGTKLTVL

SEQ ID NO: 9 - protein, 243 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPLGLQVGFDYWGQGTLVTVSTGGGGSGGGGSGGGGSLPVLTQPPSVSVSPGQTASITCSGDKLGDKYASWYQQKPGQSPVLIIYQDTKRASGIPERFSGSNSGNTATLTISGTQAVDEADYYCQAFDSSAAHFVFGAGTKLTVL

SEQ ID NO: 10 - protein, 241 aa, Homo sapiens
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVRYSYDLNFDYWGQGTLVTVSTGGGGSGGGGSGGGGSSYELMQPPSVSVSPGQTASITCSGDKLGDKYASWYQQKPGQSPVLVIYQDNKRPSGIPERFSGSNSGNTATLTISGTQAMDEADYYCQTWDSSTAVFGGGTKVTVL

SEQ ID NO: 11 - protein, 241 aa, Mus musculus
QVQLQQSGAELAKPGASVKLSCKTSGYTFTNFWMHWVKQRPGQGLEWIGYINPSSDYTKYNQKFKGKATLTADKSSSTAYMQLSSLTYEDSAVYYCARDDYSDFGFAYWGQGTLVTVSAGGGGSGGGGSGGGGSDIQMTQSPASLSASVGETVTITCRASENIYSFLAWYQQKQGKSPQLLVYNAKTLAEGVPSRFSGSGSGTQFSLKINSLQPEDFGSYYCQHHYGIPPTFGGGTKLEIK

SEQ ID NO: 12 - protein, 239 aa, Mus musculus
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCAREANWDDVDYWGQGTTLTVSSGGGGSGGGGSGGGGSQIVLTQSPAIMSASPGEKVTMTCSASSSVSYMYWYLQKPGSSPRLLIYDTSNLASGVPVRFSGSGSGTSYSLTISRMEAEDAATYYCQQWSSYPLTFGAGTKLELK

SEQ ID NO: 13 - protein, 245 aa, Mus musculus
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCAREANWDDVDYWGQGTTLTVSSGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGLYYCFQGSHVPYTFGGGTKLEIK

SEQ ID NO: 14 - protein, 248 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYSSSYDGYFDYWGQGTTLTVSSGGGGSGGGGSGGGGSDIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIK

SEQ ID NO: 15 - protein, 239 aa, Mus musculus
QVQLQQSGPELVKPGASVKISCRASGYTFTDYYIDWVKQRPGQGLEWIGWIFPGTNSTYYNEKFKGKATLTVDKSSSTAYMLLSSLTSEDSAVYFCARSGLRDFDYWGQGTTLTVSSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 16 - protein, 245 aa, Mus musculus
EVQLQQSGPVLVKPGASVKMSCKASGYTFTDYYMNWVKQSHGKGLEWIAVINPYSGGTSYNQKFKGKATLTVDKSSSTAYMELSSLTSEDSAVYYCASVSSYGNYFDYWGQGTTLTVSSGGGGSGGGGSGGGGSDIVLTQSPASLAVSLGQRATISCRASESVSIHASHLLHWYQQKPGQPPKLLIYAASNLESGVPARFSGSGSETDFTLNIHPVEEEDAATYFCQQSIEDPWTFGGGTKLEIK

SEQ ID NO: 17 - protein, 242 aa, Mus musculus
QVQLQQSGPELVKPGASVKISCKASGYTFTDYYINWVKQRPGQGLEWIGWIFPGSGSTYYNEKFKGKATLTVDKSSSTVYMLLSSLTSEDSAVYFCAREAKLGRDYFDYWGQGTTLTVSSGGGGSGGGGSGGGGSDIVMTQSHKFMSTSVGDRVSITCKASQDVSTAVAWCQQKPGQSPKLLIYSASYRYTGVPDRFTGSGSGTDFTFTISSVQAEDLAVYYCQQHYSTPYTFGGGTRLEIK

SEQ ID NO: 18 - protein, 248 aa, Mus musculus
EFQLQQSGPELVKPGASVKISCKASGYSFTDYNMNWVKQSNGKSLAWIGVINPNYGTTNYNQKFKGKATLTVDQSSSTAYMQLNSLTSEDSAVYYCARNYYGSTYDGYFDYWGQGTTLTVSSGGGGSGGGGSGGGGSDIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIT

SEQ ID NO: 19 - protein, 248 aa, Mus musculus; identical to SEQ ID NO: 18

SEQ ID NO: 20 - protein, 239 aa, Mus musculus
QVQLQQSGAELMKPGASVKISCKATGYTINGYWIEWVKERPGHGLEWIGEILPGSGSTNYNEKFKGKATFTADTSSNTAYMQLSSLTTEDSAIYYCARGMEGAMDYWGQGTSVTVSSGGGGSGGGGSGGGGSDIVMTQSHKFMSTSVGDRVSITCKASQDVSTAVAWYQKKPGQSPKLLIYSASYRYTGVPDRFTGSGSGTDFTFTISSVQAEDLAVYYCQQHYSTPPTFGGGTKLEIK

SEQ ID NO: 21 - protein, 244 aa, Mus musculus
QVQLQQPGAELVMPGASVRLSCKASGYTFTSYWMHWVKQRPGQGLEWIGEIDPSESYPNYNQNFKGKATLTVDKSSSTAYMQLSSLTSEDSAVYYCARSYYGRSGYAMDYWGQGTSVTVSSGGGGSGGGGSGGGGSNIVMTQSPKSTSMSVGERVTLNCKASENVGTYVSWYQQKPEQSPKLLIYGASNRYTGVPDRFTGSGSATDFTLTISSVQAEDLADYHCGQSYSYPPFTFGSGTKLEIK

SEQ ID NO: 22 - protein, 244 aa, Mus musculus
QVQLTESGPGLVAPSQSLSITCTVSGFSLTNYIISWVRQPPGKGLEWLGVIWTGGGTNYNSALKSRLSISKDDSKSQVFLKMNSLQTDDTARYYCARNEAVVAIFDWYFDVWGTGTTVTVSSGGGGSGGGGSGGGGSDIQMTQTTSSLSASLGDRVTISCRASQDISNYLNWYQQKPDGAVKLLIYYTSRLHSGVPSRFSGSGSGTDFSLTISNLEQEDFATYFCQQGNTLPWTFGGGTKLEIK

SEQ ID NO: 23 - protein, 248 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYGNNYDGYFDYWGQGTTLTVSSGGGGSGGGGSGGGGSDIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIT

SEQ ID NO: 24 - protein, 242 aa, Mus musculus
QIQLVQSGPELKKPGETVKISCKASGYTFTMYGMSWVKQAPGKGLKWMGWINTYSGVPTYADDFKGRFAFSLETSANTAYLQINNLKNEDTATYFCARFPYDYDGYFDVWGTGTTVTVSSGGGGSGGGGSGGGGSDIVMTQSHKFMSTSVGDRVSITCKASQDVGTAVAWYQQKPGQSPKLLIYWASTRHTGVPDRFTGSGSGTDFTLAISNVQSEDLADYFCHQFSSYPLTFGAGTRLELK

SEQ ID NO: 25 - protein, 248 aa, Mus musculus
EVQLQQSGPELVKPGASVKISCKASGYTFTDYYMNWVKQSHGKSLEWIGDINPNNGGTTYNQKFKGKATLTVDKSSSTAYMELRSLTSEDSAVYYCARRYYSSGYDGYFDVWGTGTTVTVSSGGGGSGGGGSGGGGSDIVLTQSPASLAVSLGQRATISCRASENVDNYGISFMHWYQQKPGQPPKFLIYRASNLEYGIPARFSGSGSRTDFTLTINPVETDDVATYYCQQSNKDPLTFGAGTKLELK

SEQ ID NO: 26 - protein, 244 aa, Mus musculus
EVQLQQSGPELVKPGASVKMSCKASGSTFTSYVMHWVKQKPGQGLEWIGYSNPYNDGTKYNEKFKGKATLTSDKSSSTAYMELSSLTSEDSAVYYCARLNVLYYFDNWGQGTTLTVSSGGGGSGGGGSGGGGSDIVLTQSPASLAVSLGQRATISCRASKSVSTSGYTYMHWYQQKPGQPPKLLIYLASNLESGVPARFSGSGSGTDFTLNIHPVEEEDAATYYCQHSRELPLTFGAGTKLELK

SEQ ID NO: 27 - protein, 251 aa, Mus musculus
QVQLQQPGTELVKPGASVKLSCKASGYTFTSYWMHWVKQRPGQGLEWIGNINPGNGGTDYNEKIKSKATLTVDKSTSTAYMQLSSLTSEDSAVYYCARGGGYYGYDGYWYFDVWGTGTTVTVSSGGGGSGGGGSGGGGSDIVMTQAAFSIPVTLGTSTSISCRSTKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 28 - protein, 243 aa, Mus musculus
EVQLQQSGPVLVKPGPSVKISCEASGFTFTDYYMHWVKQNHGKSLEWIGLVYPYNGDTIYNQKFKGKATLTVDTSSSTAYMDLHSLTSEDSAVYYCARGANWGDYWGQGTTLTVSSGGGGSGGGGSGGGGSDVVMTQTPLSLPVSLGDQASISCRSSQSLVHSNGNTYLHWFLQKPGQSPKLLIYKVSNRFSGVPDRFSGSGSGTDFTLKISRVEAEDLGLYFCSQSTHVPPTFGGGTKLEIK

SEQ ID NO: 29 - protein, 241 aa, Mus musculus
QVQLQQPGTELVKPGASVKLSCKASGYTFTSYWMHWVKQRPGQGLEWIGNISPSNGGTNYNENFKSKATLTVDKSSSTAYMQLSSLTSEDSAVYYCATYYVDYWGQGTTLTVSSGGGGSGGGGSGGGGSDVVMTQTPLTLSVTIGQPASISCKSTQSLLDSDGKTYLNWFLQRPGQSPKRLIYLVSKLDSGVPDRFTGSGSGTDFTLKISRVEAEDLGLYYCWQGTHFPQTFGGGTKLEIK

SEQ ID NO: 30 - protein, 243 aa, Mus musculus
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRVYGYDPYAMNYWGPGTSVTVSSGGGGSGGGGSGGGGSQIVLTQSPALMSASPGEKVTMTCSASSSVSYMYWYQQKPRSSPKPWIYLTSTLASGVPARFSGSGSGTSYSLTISSMEAEDAATYYCQQWSSNPYTFGGGTKLEIK

SEQ ID NO: 31 - protein, 248 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAPHYYGSSYDWYFDVWGTGTTVTVSSGGGGSGGGGSGGGGSDIVLTQSPASLAVSLGQRATISCRASESVDNYGISFMHWYQQKPGQPPKLLIYRASNLESGIPARFSGSGSRTDFTLTINPVETDDVATYYCQQSNKDPLTFGAGTKLELK

SEQ ID NO: 32 - protein, 242 aa, Mus musculus
EVQLQQSGPELVKPGASVKIPCKASGYTFTDYNMDWVKQSHGKSLEWIGDINPNNGGTIYNQKFKGKATLTVDKSSSTAYMELRSLTSEDTAVYYCAREDDSRYWYFDVWGTGTTVTVSSGGGGSGGGGSGGGGSDIVMTQSHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLEIK

SEQ ID NO: 33 - protein, 240 aa, Mus musculus
QIQFVQSGPELKKPGETVKISCKASVYTFTEYPMHWVKQAPGKGFKWMGWINTYSGEPTYADDFKGRFAFSLETSASTAYLQINNLKNEDTASYFCAREGWLDAMDYWGQGTSVTVSSGGGGSGGGGSGGGGSDIVMTQSQKFMSTIVGDRVSITCKASQNVGTAVAWYQQKPGQSPKLLIYSASNRYTGVPDRFTGSGSGTDFTLTISNMQSEDLADYFCQQYSSYTWTFGGGTKLEIK

SEQ ID NO: 34 - protein, 237 aa, Mus musculus
QVQLKQSGPGLVQPSQSLSITCTVSGFSLTTFGVHWVRQSPGKGLEWLGVIWSGGSTDYNAAFISRLSISKDNSKSQVFFKMNSLQVDDTAIYYCAPRLGLRAYWGQGTLVTVSAGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEVK

SEQ ID NO: 35 - protein, 239 aa, Mus musculus
QVQLKQSGPGLVQPSQSLSITCTVSGFSLTSYGVHWVRQSPGKGLEWLGVIWSGGTTDYNAAFISRLSISKDNSKSQVFFKMNSLQADDTAIYYCARMGGTGYFDVWGTGTTVTVSSGGGGSGGGGSGGGGSNIVMTQSPKSMSMSVGERVTLSCKAGENVGPYVSWYQQKPEQSPKLLIYGASNRFTGVPDRFTGSGSATDFTLTISSVQAEDLADYHCGQSYSYPFTFGSGTKLEIK

SEQ ID NO: 36 - protein, 244 aa, Mus musculus
QVQLKQSGPGLVQPSQSLSITCTVSGFSLTSYGVHWVRQSPGKGLEWLGVIWSGGTTDYNAAFISRLSISKDNSKSQVFFKMNSLQADDTAIYYCARMGGTGYFDVWGTGTTVTVSSGGGGSGGGGSGGGGSDIVMTQAAFSNPVTLGTSASISCRSSKSLLHSNGITYLYWYLQKPGLSPQLLIYHMSNLASGVPDRFSSSGSGTDFTLRISRVEAEDVGVYYCAQNLELPWTFGGGTKLEIK

SEQ ID NO: 37 - protein, 238 aa, Mus musculus
QVQLVQSGAEVKKPGASVKVSCKASGYTFTDYNMHWVRQAPGQGLEWMGYIYPYNGGTAYNQKFKNRVTMTRDTSTSTVYMELSSLRSEDTAVYYCARGLDVMDYWGQGTLVTVSSGGGGSGGGGSGGGGSAIQLTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIK

SEQ ID NO: 38 - protein, 238 aa, Mus musculus
DIQMTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIKGGGGSGGGGSGGGGSQVQLVQSGAEVKKPGASVKVSCKVSGYTFTDYNMHWVRQAPGKGLEWMGYIYPYNGGTAYNQKFKNRVTMTEDTSTDTAYMELSSLRSEDTAVYYCARGLDVMDYWGQGTLVTVSS

SEQ ID NO: 39 - protein, 120 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVST

SEQ ID NO: 40 - protein, 120 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVST

SEQ ID NO: 41 - protein, 120 aa, Homo sapiens
QLQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVST

SEQ ID NO: 42 - protein, 120 aa, Homo sapiens
QMQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKPSRGYSRSLDYWGQGTLVTVST

SEQ ID NO: 43 - protein, 120 aa, Homo sapiens
QVQLVESGAEVKKPGESLKISCKGSGYSFTSYWIGWVRQMPGKGLEWMGIIYPGDSDTRYSPSFQGQVTISADKSISTAYLQWSSLKASDTAMYYCARLQSGWLHAFDIWGQGTMVTVST

SEQ ID NO: 44 - protein, 125 aa, Homo sapiens
QVQLLQSAAEVKKPGESLKISCKGSGYSFTSYWIGWVRQMPGKGLEWMGIIYPGDSDTRYSPSFQGQITISADKSISTAYLQWSSLKASDTAMYYCARLKWSGLSHYYYYYMDVWGKGTTVTVST

SEQ ID NO: 45 - protein, 125 aa, Homo sapiens
QVQLLQSAAEVKKPGESLKISCKGSGYSFTSYWIGWVRQMPGKGLEWMGIIYPGDSDTRYSPSFQGQVTISADKSISTAYLQWSSLKASDTAMYYCARLKWSGLSHYYYYYMDVWGKGTTVTVST

SEQ ID NO: 46 - protein, 125 aa, Homo sapiens; identical to SEQ ID NO: 45

SEQ ID NO: 47 - protein, 120 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPLGLQVGFDYWGQGTLVTVST

SEQ ID NO: 48 - protein, 120 aa, Homo sapiens
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVRYSYDLNFDYWGQGTLVTVST

SEQ ID NO: 49 - protein, 119 aa, Mus musculus
QVQLQQSGAELAKPGASVKLSCKTSGYTFTNFWMHWVKQRPGQGLEWIGYINPSSDYTKYNQKFKGKATLTADKSSSTAYMQLSSLTYEDSAVYYCARDDYSDFGFAYWGQGTLVTVSA

SEQ ID NO: 50 - protein, 118 aa, Mus musculus
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCAREANWDDVDYWGQGTTLTVSS

SEQ ID NO: 51 - protein, 118 aa, Mus musculus; identical to SEQ ID NO: 50

SEQ ID NO: 52 - protein, 122 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYSSSYDGYFDYWGQGTTLTVSS

SEQ ID NO: 53 - protein, 117 aa, Mus musculus
QVQLQQSGPELVKPGASVKISCRASGYTFTDYYIDWVKQRPGQGLEWIGWIFPGTNSTYYNEKFKGKATLTVDKSSSTAYMLLSSLTSEDSAVYFCARSGLRDFDYWGQGTTLTVSS

SEQ ID NO: 54 - protein, 119 aa, Mus musculus
EVQLQQSGPVLVKPGASVKMSCKASGYTFTDYYMNWVKQSHGKGLEWIAVINPYSGGTSYNQKFKGKATLTVDKSSSTAYMELSSLTSEDSAVYYCASVSSYGNYFDYWGQGTTLTVSS

SEQ ID NO: 55 - protein, 120 aa, Mus musculus
QVQLQQSGPELVKPGASVKISCKASGYTFTDYYINWVKQRPGQGLEWIGWIFPGSGSTYYNEKFKGKATLTVDKSSSTVYMLLSSLTSEDSAVYFCAREAKLGRDYFDYWGQGTTLTVSS

SEQ ID NO: 56 - protein, 120 aa, Mus musculus
QIQLVQSGPELKKPGETVKISCKASGYTFTTYGMSWVKQAPGKGLKWMGWINTYSGVPTYADDFKGRFAFSLETSASTAYLQINNLKNEDTTTYFCARFPYDFDGYFDVWGTGTAVTVSS

SEQ ID NO: 57 - protein, 122 aa, Mus musculus
EFQLQQSGPELVKPGASVKISCKASGYSFTDYNMNWVKQSNGKSLAWIGVINPNYGTTNYNQKFKGKATLTVDQSSSTAYMQLNSLTSEDSAVYYCARNYYGSTYDGYFDYWGQGTTLTVSS

SEQ ID NO: 58 - protein, 117 aa, Mus musculus
QVQLQQSGAELMKPGASVKISCKATGYTINGYWIEWVKERPGHGLEWIGEILPGSGSTNYNEKFKGKATFTADTSSNTAYMQLSSLTTEDSAIYYCARGMEGAMDYWGQGTSVTVSS

SEQ ID NO: 59 - protein, 121 aa, Mus musculus
QVQLQQPGAELVMPGASVRLSCKASGYTFTSYWMHWVKQRPGQGLEWIGEIDPSESYPNYNQNFKGKATLTVDKSSSTAYMQLSSLTSEDSAVYYCARSYYGRSGYAMDYWGQGTSVTVSS

SEQ ID NO: 60 - protein, 122 aa, Mus musculus
QVQLTESGPGLVAPSQSLSITCTVSGFSLTNYIISWVRQPPGKGLEWLGVIWTGGGTNYNSALKSRLSISKDDSKSQVFLKMNSLQTDDTARYYCARNEAVVAIFDWYFDVWGTGTTVTVSS

SEQ ID NO: 61 - protein, 122 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYGNNYDGYFDYWGQGTTLTVSS

SEQ ID NO: 62 - protein, 120 aa, Mus musculus
QIQLVQSGPELKKPGETVKISCKASGYTFTMYGMSWVKQAPGKGLKWMGWINTYSGVPTYADDFKGRFAFSLETSANTAYLQINNLKNEDTATYFCARFPYDYDGYFDVWGTGTTVTVSS

SEQ ID NO: 63 - protein, 122 aa, Mus musculus
EVQLQQSGPELVKPGASVKISCKASGYTFTDYYMNWVKQSHGKSLEWIGDINPNNGGTTYNQKFKGKATLTVDKSSSTAYMELRSLTSEDSAVYYCARRYYSSGYDGYFDVWGTGTTVTVSS

SEQ ID NO: 64 - protein, 118 aa, Mus musculus
EVQLQQSGPELVKPGASVKMSCKASGSTFTSYVMHWVKQKPGQGLEWIGYSNPYNDGTKYNEKFKGKATLTSDKSSSTAYMELSSLTSEDSAVYYCARLNVLYYFDNWGQGTTLTVSS

SEQ ID NO: 65 - protein, 124 aa, Mus musculus
QVQLQQPGTELVKPGASVKLSCKASGYTFTSYWMHWVKQRPGQGLEWIGNINPGNGGTDYNEKIKSKATLTVDKSTSTAYMQLSSLTSEDSAVYYCARGGGYYGYDGYWYFDVWGTGTTVTVSS

SEQ ID NO: 66 - protein, 116 aa, Mus musculus
EVQLQQSGPVLVKPGPSVKISCEASGFTFTDYYMHWVKQNHGKSLEWIGLVYPYNGDTIYNQKFKGKATLTVDTSSSTAYMDLHSLTSEDSAVYYCARGANWGDYWGQGTTLTVSS

SEQ ID NO: 67 - protein, 114 aa, Mus musculus
QVQLQQPGTELVKPGASVKLSCKASGYTFTSYWMHWVKQRPGQGLEWIGNISPSNGGTNYNENFKSKATLTVDKSSSTAYMQLSSLTSEDSAVYYCATYYVDYWGQGTTLTVSS

SEQ ID NO: 68 - protein, 122 aa, Mus musculus
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRVYGYDPYAMNYWGPGTSVTVSS

SEQ ID NO: 69 - protein, 122 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAPHYYGSSYDWYFDVWGTGTTVTVSS

SEQ ID NO: 70 - protein, 120 aa, Mus musculus
EVQLQQSGPELVKPGASVKIPCKASGYTFTDYNMDWVKQSHGKSLEWIGDINPNNGGTIYNQKFKGKATLTVDKSSSTAYMELRSLTSEDTAVYYCAREDDSRYWYFDVWGTGTTVTVSS

SEQ ID NO: 71 - protein, 118 aa, Mus musculus
QIQFVQSGPELKKPGETVKISCKASVYTFTEYPMHWVKQAPGKGFKWMGWINTYSGEPTYADDFKGRFAFSLETSASTAYLQINNLKNEDTASYFCAREGWLDAMDYWGQGTSVTVSS

SEQ ID NO: 72 - protein, 115 aa, Mus musculus
QVQLKQSGPGLVQPSQSLSITCTVSGFSLTTFGVHWVRQSPGKGLEWLGVIWSGGSTDYNAAFISRLSISKDNSKSQVFFKMNSLQVDDTAIYYCAPRLGLRAYWGQGTLVTVSA

SEQ ID NO: 73 - protein, 117 aa, Mus musculus
QVQLKQSGPGLVQPSQSLSITCTVSGFSLTSYGVHWVRQSPGKGLEWLGVIWSGGTTDYNAAFISRLSISKDNSKSQVFFKMNSLQADDTAIYYCARMGGTGYFDVWGTGTTVTVSS

SEQ ID NO: 74 - protein, 117 aa, Mus musculus; identical to SEQ ID NO: 73

SEQ ID NO: 75 - protein, 116 aa, Mus musculus
QVQLVQSGAEVKKPGASVKVSCKASGYTFTDYNMHWVRQAPGQGLEWMGYIYPYNGGTAYNQKFKNRVTMTRDTSTSTVYMELSSLRSEDTAVYYCARGLDVMDYWGQGTLVTVSS

SEQ ID NO: 76 - protein, 116 aa, Mus musculus
QVQLVQSGAEVKKPGASVKVSCKVSGYTFTDYNMHWVRQAPGKGLEWMGYIYPYNGGTAYNQKFKNRVTMTEDTSTDTAYMELSSLRSEDTAVYYCARGLDVMDYWGQGTLVTVSS

SEQ ID NO: 77 - protein, 106 aa, Homo sapiens
SYELMQPPSVSVSPGQTASITCSGDKLGDKYVSWYQQKPGQSPVLVIYQDTKRPSGIPERFSGSNSGNTATLTISGTQAMDEADYYCQAWDSGTAIFGGGTKVTVL

SEQ ID NO: 78 - protein, 106 aa, Homo sapiens
QAGLTQPPSVSVSPGQTASITCSGDKLGDKYVSWYQQKPGQSPVLVIYQDSKRPSGIPERFSGSNSGNTATLTISGTQAMDEADYYCQAWDSSTVVFGGGTKVTVL

SEQ ID NO: 79 - protein, 106 aa, Homo sapiens
QAGLTQPPSVSVSPGQTASITCFGDKLGHKYVSWYQQKPGQSPVLVIYQDSKRPSGIPERFSGSNSGNTATLTISGTQAMDEADYYCQAWDSSTVVFGGGTKLTVL

SEQ ID NO: 80 - protein, 108 aa, Homo sapiens
QPVLTQPPSVSVSPGQTASITCSGDKLGDKFTSWYQQRPGQSPVLVIYQDNKRPSGIPERFSGSNSGNTATLTISRVEAGDEADYYCQVWDSSSDHWVFGGGTQLTVL

SEQ ID NO: 81 - protein, 106 aa, Homo sapiens
QSVLTQPPSVSVSPGQTARISCSGDKLGDKYVSWYQQKPGQSPVLVIYEDSKRPSGIPERLSGSNSGNTATLTISGTQAMDEADYYCQAWDSSTVVFGGGTKLTVL

SEQ ID NO: 82 - protein, 109 aa, Homo sapiens
LSELTQDPAVSVALGQTVRITCQGDSLRNYYASWYQQKPGQAPVLVIYGKNNRPSGIPDRFSGSRSGNTASLTITGAQAEDEADYYCNSRDSSGNHPVVFGGGTKLTVL

SEQ ID NO: 83 - protein, 108 aa, Homo sapiens
LSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTKLTVL

SEQ ID NO: 84 - protein, 108 aa, Homo sapiens
LSELTQDPAVSVALGQTVRITCQGDSLRSYYASWYQQKPGQAPVLVIYGKNNRPSGIPDRFSGSSSGNTASLTITGAQAEDEADYYCNSRDSSGNHVIFGGGTKLTVL

SEQ ID NO: 85 - protein, 108 aa, Homo sapiens
LPVLTQPPSVSVSPGQTASITCSGDKLGDKYASWYQQKPGQSPVLIIYQDTKRASGIPERFSGSNSGNTATLTISGTQAVDEADYYCQAFDSSAAHFVFGAGTKLTVL

SEQ ID NO: 86 - protein, 106 aa, Homo sapiens
SYELMQPPSVSVSPGQTASITCSGDKLGDKYASWYQQKPGQSPVLVIYQDNKRPSGIPERFSGSNSGNTATLTISGTQAMDEADYYCQTWDSSTAVFGGGTKVTVL

SEQ ID NO: 87 - protein, 107 aa, Mus musculus
DIQMTQSPASLSASVGETVTITCRASENIYSFLAWYQQKQGKSPQLLVYNAKTLAEGVPSRFSGSGSGTQFSLKINSLQPEDFGSYYCQHHYGIPPTFGGGTKLEIK

SEQ ID NO: 88 - protein, 106 aa, Mus musculus
QIVLTQSPAIMSASPGEKVTMTCSASSSVSYMYWYLQKPGSSPRLLIYDTSNLASGVPVRFSGSGSGTSYSLTISRMEAEDAATYYCQQWSSYPLTFGAGTKLELK

SEQ ID NO: 89 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGLYYCFQGSHVPYTFGGGTKLEIK

SEQ ID NO: 90 - protein, 111 aa, Mus musculus
DIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIK

SEQ ID NO: 91 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 92 - protein, 111 aa, Mus musculus
DIVLTQSPASLAVSLGQRATISCRASESVSIHASHLLHWYQQKPGQPPKLLIYAASNLESGVPARFSGSGSETDFTLNIHPVEEEDAATYFCQQSIEDPWTFGGGTKLEIK

SEQ ID NO: 93 - protein, 107 aa, Mus musculus
DIVMTQSHKFMSTSVGDRVSITCKASQDVSTAVAWCQQKPGQSPKLLIYSASYRYTGVPDRFTGSGSGTDFTFTISSVQAEDLAVYYCQQHYSTPYTFGGGTRLEIK

SEQ ID NO: 94 - protein, 107 aa, Mus musculus
DIVMTQSHKFMSTSVGDRVSITCKASQDVGTAVAWYQEKPGQSPKILIYWASTRHTGVPDRFTGSGSGTDFTLTISNVQSEDLADYFCQQYSSYPLTFGAGTKLELK

SEQ ID NO: 95 - protein, 111 aa, Mus musculus
DIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIT

SEQ ID NO: 96 - protein, 107 aa, Mus musculus
DIVMTQSHKFMSTSVGDRVSITCKASQDVSTAVAWYQKKPGQSPKLLIYSASYRYTGVPDRFTGSGSGTDFTFTISSVQAEDLAVYYCQQHYSTPPTFGGGTKLEIK

SEQ ID NO: 97 - protein, 108 aa, Mus musculus
NIVMTQSPKSTSMSVGERVTLNCKASENVGTYVSWYQQKPEQSPKLLIYGASNRYTGVPDRFTGSGSATDFTLTISSVQAEDLADYHCGQSYSYPPFTFGSGTKLEIK

SEQ ID NO: 98 - protein, 107 aa, Mus musculus
DIQMTQTTSSLSASLGDRVTISCRASQDISNYLNWYQQKPDGAVKLLIYYTSRLHSGVPSRFSGSGSGTDFSLTISNLEQEDFATYFCQQGNTLPWTFGGGTKLEIK

SEQ ID NO: 99 - protein, 111 aa, Mus musculus; identical to SEQ ID NO: 95

SEQ ID NO: 100 - protein, 107 aa, Mus musculus
DIVMTQSHKFMSTSVGDRVSITCKASQDVGTAVAWYQQKPGQSPKLLIYWASTRHTGVPDRFTGSGSGTDFTLAISNVQSEDLADYFCHQFSSYPLTFGAGTRLELK

SEQ ID NO: 101 - protein, 111 aa, Mus musculus
DIVLTQSPASLAVSLGQRATISCRASENVDNYGISFMHWYQQKPGQPPKFLIYRASNLEYGIPARFSGSGSRTDFTLTINPVETDDVATYYCQQSNKDPLTFGAGTKLELK

SEQ ID NO: 102 - protein, 111 aa, Mus musculus
DIVLTQSPASLAVSLGQRATISCRASKSVSTSGYTYMHWYQQKPGQPPKLLIYLASNLESGVPARFSGSGSGTDFTLNIHPVEEEDAATYYCQHSRELPLTFGAGTKLELK

SEQ ID NO: 103 - protein, 112 aa, Mus musculus
DIVMTQAAFSIPVTLGTSTSISCRSTKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 104 - protein, 112 aa, Mus musculus
DVVMTQTPLSLPVSLGDQASISCRSSQSLVHSNGNTYLHWFLQKPGQSPKLLIYKVSNRFSGVPDRFSGSGSGTDFTLKISRVEAEDLGLYFCSQSTHVPPTFGGGTKLEIK

SEQ ID NO: 105 - protein, 112 aa, Mus musculus
DVVMTQTPLTLSVTIGQPASISCKSTQSLLDSDGKTYLNWFLQRPGQSPKRLIYLVSKLDSGVPDRFTGSGSGTDFTLKISRVEAEDLGLYYCWQGTHFPQTFGGGTKLEIK

SEQ ID NO: 106 - protein, 106 aa, Mus musculus
QIVLTQSPALMSASPGEKVTMTCSASSSVSYMYWYQQKPRSSPKPWIYLTSTLASGVPARFSGSGSGTSYSLTISSMEAEDAATYYCQQWSSNPYTFGGGTKLEIK

SEQ ID NO: 107 - protein, 111 aa, Mus musculus
DIVLTQSPASLAVSLGQRATISCRASESVDNYGISFMHWYQQKPGQPPKLLIYRASNLESGIPARFSGSGSRTDFTLTINPVETDDVATYYCQQSNKDPLTFGAGTKLELK

SEQ ID NO: 108 - protein, 107 aa, Mus musculus
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLEIK

SEQ ID NO: 109 - protein, 107 aa, Mus musculus
DIVMTQSQKFMSTIVGDRVSITCKASQNVGTAVAWYQQKPGQSPKLLIYSASNRYTGVPDRFTGSGSGTDFTLTISNMQSEDLADYFCQQYSSYTWTFGGGTKLEIK

SEQ ID NO: 110 - protein, 107 aa, Mus musculus
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEVK

SEQ ID NO: 111 - protein, 107 aa, Mus musculus
NIVMTQSPKSMSMSVGERVTLSCKAGENVGPYVSWYQQKPEQSPKLLIYGASNRFTGVPDRFTGSGSATDFTLTISSVQAEDLADYHCGQSYSYPFTFGSGTKLEIK

SEQ ID NO: 112 - protein, 112 aa, Mus musculus
DIVMTQAAFSNPVTLGTSASISCRSSKSLLHSNGITYLYWYLQKPGLSPQLLIYHMSNLASGVPDRFSSSGSGTDFTLRISRVEAEDVGVYYCAQNLELPWTFGGGTKLEIK

SEQ ID NO: 113 - protein, 107 aa, Mus musculus
AIQLTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIK

SEQ ID NO: 114 - protein, 107 aa, Mus musculus
DIQMTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIK

SEQ ID NO: 115 - protein, 10 aa, Homo sapiens
GYSFTSYWIG

SEQ ID NO: 116 - protein, 10 aa, Homo sapiens
GFTFSSYGMH

SEQ ID NO: 117 - protein, 10 aa, Mus musculus
GYTFTNFWMH

SEQ ID NO: 118 - protein, 10 aa, Mus musculus
GYTFTSYTMH

SEQ ID NO: 119 - protein, 10 aa, Mus musculus
GYSFTDYNMN

SEQ ID NO: 120 - protein, 10 aa, Mus musculus
GYTFTDYYID

SEQ ID NO: 121 - protein, 10 aa, Mus musculus
GYTFTDYYMN

SEQ ID NO: 122 - protein, 10 aa, Mus musculus
GYTFTDYYIN

SEQ ID NO: 123 - protein, 12 aa, Mus musculus
GYTFTTYGMSWV

SEQ ID NO: 124 - protein, 7 aa, Mus musculus
GYTINGY

SEQ ID NO: 125 - protein, 10 aa, Mus musculus
GYTFTSYWMH

SEQ ID NO: 126 - protein, 10 aa, Mus musculus
GFSLTNYIIS

SEQ ID NO: 127 - protein, 12 aa, Mus musculus
GYTFTMYGMSWV

SEQ ID NO: 128 - protein, 10 aa, Mus musculus
GSTFTSYVMH

SEQ ID NO: 129 - protein, 10 aa, Mus musculus
GFTFTDYYMH

SEQ ID NO: 130 - protein, 12 aa, Mus musculus
GFSLSTSGMGVS

SEQ ID NO: 131 - protein, 10 aa, Mus musculus
GYTFTDYNMD

SEQ ID NO: 132 - protein, 12 aa, Mus musculus
VYTFTEYPMHWV

SEQ ID NO: 133 - protein, 10 aa, Mus musculus
GFSLTTFGVH

SEQ ID NO: 134 - protein, 10 aa, Mus musculus
GFSLTSYGVH

SEQ ID NO: 135 - protein, 10 aa, Mus musculus
GYTFTDYNMH

SEQ ID NO: 136 - protein, 17 aa, Homo sapiens
IIYPGDSDTRYSPSFQG

SEQ ID NO: 137 - protein, 17 aa, Homo sapiens
FIRYDGSNKYYADSVKG

SEQ ID NO: 138 - protein, 16 aa, Homo sapiens
INPSSDYTKYNQKFKG

SEQ ID NO: 139 - protein, 16 aa, Homo sapiens
INPSSGYTKYNQKFKD

SEQ ID NO: 140 - protein, 16 aa, Mus musculus
INPNYGTTSYNQKFKG

SEQ ID NO: 141 - protein, 16 aa, Mus musculus
IFPGTNSTYYNEKFKG

SEQ ID NO: 142 - protein, 16 aa, Mus musculus
INPYSGGTSYNQKFKG

SEQ ID NO: 143 - protein, 16 aa, Mus musculus
IFPGSGSTYYNEKFKG

SEQ ID NO: 144 - protein, 16 aa, Mus musculus
WINTYSGVPTYADDFK

SEQ ID NO: 145 - protein, 16 aa, Mus musculus
INPNYGTTNYNQKFKG

SEQ ID NO: 146 - protein, 16 aa, Mus musculus
ILPGSGSTNYNEKFKG

SEQ ID NO: 147 - protein, 16 aa, Mus musculus
IDPSESYPNYNQNFKG

SEQ ID NO: 148 - protein, 15 aa, Mus musculus
IWTGGGTNYNSALKS

SEQ ID NO: 149 - protein, 16 aa, Mus musculus
INPNNGGTTYNQKFKG

SEQ ID NO: 150 - protein, 16 aa, Mus musculus
SNPYNDGTKYNEKFKG

SEQ ID NO: 151 - protein, 16 aa, Mus musculus
INPGNGGTDYNEKIKS

SEQ ID NO: 152 - protein, 16 aa, Mus musculus
VYPYNGDTIYNQKFKG

SEQ ID NO: 153 - protein, 16 aa, Mus musculus
ISPSNGGTNYNENFKS

SEQ ID NO: 154 - protein, 15 aa, Mus musculus
IYWDDDKRYNPSLKS

SEQ ID NO: 155 - protein, 16 aa, Mus musculus
INPNNGGTSYNQKFKG

SEQ ID NO: 156 - protein, 16 aa, Mus musculus
INPNNGGTIYNQKFKG

SEQ ID NO: 157 - protein, 16 aa, Mus musculus
WINTYSGEPTYADDFK

SEQ ID NO: 158 - protein, 15 aa, Mus musculus
IWSGGSTDYNAAFIS

SEQ ID NO: 159 - protein, 15 aa, Mus musculus
IWSGGTTDYNAAFIS

SEQ ID NO: 160 - protein, 17 aa, Mus musculus
YIYPYNGGTAYNQKFKN

SEQ ID NO: 161 - protein, 13 aa, Homo sapiens
ARYIQGLGYYFDY

SEQ ID NO: 162 - protein, 13 aa, Homo sapiens
AKPSRGYSRSLDY

SEQ ID NO: 163 - protein, 13 aa, Homo sapiens
ARLQSGWLHAFDI

SEQ ID NO: 164 - protein, 18 aa, Homo sapiens
ARLKWSGLSHYYYYYMDV

SEQ ID NO: 165 - protein, 13 aa, Homo sapiens
ARLPLGLQVGFDY

SEQ ID NO: 166 - protein, 13 aa, Mus musculus
ARVRYSYDLNFDY

SEQ ID NO: 167 - protein, 12 aa, Mus musculus
ARDDYSDFGFAY

SEQ ID NO: 168 - protein, 11 aa, Mus musculus
AREANWDDVDY

SEQ ID NO: 169 - protein, 15 aa, Mus musculus
ARNYYSSSYDGYFDY

SEQ ID NO: 170 - protein, 10 aa, Mus musculus
ARSGLRDFDY

SEQ ID NO: 171 - protein, 12 aa, Mus musculus
ASVSSYGNYFDY

SEQ ID NO: 172 - protein, 13 aa, Mus musculus
AREAKLGRDYFDY

SEQ ID NO: 173 - protein, 13 aa, Mus musculus
ARFPYDFDGYFDV

SEQ ID NO: 174 - protein, 15 aa, Mus musculus
ARNYYGSTYDGYFDY

SEQ ID NO: 175 - protein, 10 aa, Mus musculus
ARGMEGAMDY

SEQ ID NO: 176 - protein, 14 aa, Mus musculus
ARSYYGRSGYAMDY

SEQ ID NO: 177 - protein, 16 aa, Mus musculus
ARNEAVVAIFDWYFDV

SEQ ID NO: 178 - protein, 15 aa, Mus musculus
ARNYYGNNYDGYFDY

SEQ ID NO: 179 - protein, 13 aa, Mus musculus
ARFPYDYDGYFDV

SEQ ID NO: 180 - protein, 15 aa, Mus musculus
ARRYYSSGYDGYFDV

SEQ ID NO: 181 - protein, 11 aa, Mus musculus
ARLNVLYYFDN

SEQ ID NO: 182 - protein, 17 aa, Mus musculus
ARGGGYYGYDGYWYFDV

SEQ ID NO: 183 - protein, 9 aa, Mus musculus
ARGANWGDY

SEQ ID NO: 184 - protein, 7 aa, Mus musculus
ATYYVDY

SEQ ID NO: 185 - protein, 14 aa, Mus musculus
ARRVYGYDPYAMNY

SEQ ID NO: 186 - protein, 15 aa, Mus musculus
APHYYGSSYDWYFDV

SEQ ID NO: 187 - protein, 13 aa, Mus musculus
AREDDSRYWYFDV

SEQ ID NO: 188 - protein, 11 aa, Mus musculus
AREGWLDAMDY

SEQ ID NO: 189 - protein, 9 aa, Mus musculus
APRLGLRAY

SEQ ID NO: 190 - protein, 11 aa, Mus musculus
ARMGGTGYFDV

SEQ ID NO: 191 - protein, 9 aa, Mus musculus
ARGLDVMDY

SEQ ID NO: 192 - protein, 11 aa, Homo sapiens
SGDKLGDKYVS

SEQ ID NO: 193 - protein, 11 aa, Homo sapiens
FGDKLGHKYVS

SEQ ID NO: 194 - protein, 11 aa, Homo sapiens
SGDKLGDKFTS

SEQ ID NO: 195 - protein, 11 aa, Homo sapiens
QGDSLRNYYAS

SEQ ID NO: 196 - protein, 11 aa, Homo sapiens
QGDSLRSYYAS

SEQ ID NO: 197 - protein, 11 aa, Homo sapiens
SGDKLGDKYAS

SEQ ID NO: 198 - protein, 9 aa, Mus musculus
NIYSFLAWY

SEQ ID NO: 199 - protein, 8 aa, Mus musculus
SVSYMYWY

SEQ ID NO: 200 - protein, 14 aa, Mus musculus
SIVHSNGNTYLEWY

SEQ ID NO: 201 - protein, 13 aa, Mus musculus
SVDNYGISFMHWY

SEQ ID NO: 202 - protein, 9 aa, Mus musculus
SISDYLHWY

SEQ ID NO: 203 - protein, 14 aa, Mus musculus
SVSIHASHLLHWYQ

SEQ ID NO: 204 - protein, 9 aa, Mus musculus
DVSTAVAWC

SEQ ID NO: 205 - protein, 9 aa, Mus musculus
DVGTAVAWY

SEQ ID NO: 206 - protein, 9 aa, Mus musculus
DVSTAVAWY

SEQ ID NO: 207 - protein, 9 aa, Mus musculus
NVGTYVSWY

SEQ ID NO: 208 - protein, 9 aa, Mus musculus
DISNYLNWY

SEQ ID NO: 209 - protein, 13 aa, Mus musculus
NVDNYGISFMHWY

SEQ ID NO: 210 - protein, 13 aa, Mus musculus
SVSTSGYTYMHWY

SEQ ID NO: 211 - protein, 14 aa, Mus musculus
SLLHSNGITYLYWY

SEQ ID NO: 212 - protein, 14 aa, Mus musculus
SLVHSNGNTYLHWF

SEQ ID NO: 213 - protein, 14 aa, Mus musculus
SLLDSDGKTYLNWF

SEQ ID NO: 214 - protein, 9 aa, Mus musculus
NVGTAVAWY

SEQ ID NO: 215 - protein, 9 aa, Mus musculus
DINSYLSWF

SEQ ID NO: 216 - protein, 9 aa, Mus musculus
NVGPYVSWY

SEQ ID NO: 217 - protein, 11 aa, Mus musculus
RASENIYSYLA

SEQ ID NO: 218 - protein, 7 aa, Homo sapiens
QDTKRPS

SEQ ID NO: 219 - protein, 7 aa, Homo sapiens
QDSKRPS

SEQ ID NO: 220 - protein, 7 aa, Homo sapiens
QDNKRPS

SEQ ID NO: 221 - protein, 7 aa, Homo sapiens
EDSKRPS

SEQ ID NO: 222 - protein, 7 aa, Homo sapiens
GKNNRPS

SEQ ID NO: 223 - protein, 7 aa, Homo sapiens
QDTKRAS

SEQ ID NO: 224 - protein, 9 aa, Mus musculus
KTLAEGVPS

SEQ ID NO: 225 - protein, 8 aa, Mus musculus
NLASGVPV

SEQ ID NO: 226 - protein, 9 aa, Mus musculus
SNRFSGVPD

SEQ ID NO: 227 - protein, 8 aa, Mus musculus
NLESGIPA

SEQ ID NO: 228 - protein, 8 aa, Mus musculus
QSISGIPS

SEQ ID NO: 229 - protein, 8 aa, Mus musculus
NLESGVPA

SEQ ID NO: 230 - protein, 8 aa, Mus musculus
YRYTGVPD

SEQ ID NO: 231 - protein, 8 aa, Mus musculus
TRHTGVPD

SEQ ID NO: 232 - protein, 8 aa, Mus musculus
NRYTGVPD

SEQ ID NO: 233 - protein, 9 aa, Mus musculus
SRLHSGVPS

SEQ ID NO: 234 - protein, 8 aa, Mus musculus
NLEYGIPA

SEQ ID NO: 235 - protein, 8 aa, Mus musculus
NLASGVPD

SEQ ID NO: 236 - protein, 9 aa, Mus musculus
SKLDSGVPD

SEQ ID NO: 237 - protein, 8 aa, Mus musculus
TLASGVPA

SEQ ID NO: 238 - protein, 9 aa, Mus musculus
NRLVDGVPS

SEQ ID NO: 239 - protein, 8 aa, Mus musculus
NRFTGVPD

SEQ ID NO: 240 - protein, 7 aa, Mus musculus
NAKILAE

SEQ ID NO: 241 - protein, 9 aa, Homo sapiens
QAWDSGTAI

SEQ ID NO: 242 - protein, 9 aa, Homo sapiens
QAWDSSTVV

SEQ ID NO: 243 - protein, 11 aa, Homo sapiens
QVWDSSSDHWV

SEQ ID NO: 244 - protein, 12 aa, Homo sapiens
NSRDSSGNHPVV

SEQ ID NO: 245 - protein, 11 aa, Homo sapiens
NSRDSSGNHLV

SEQ ID NO: 246 - protein, 11 aa, Homo sapiens
NSRDSSGNHVI

SEQ ID NO: 247 - protein, 11 aa, Homo sapiens
QAFDSSAAHFV

SEQ ID NO: 248 - protein, 9 aa, Homo sapiens
QTWDSSTAV

SEQ ID NO: 249 - protein, 9 aa, Mus musculus
QHHYGIPPT

SEQ ID NO: 250 - protein, 9 aa, Mus musculus
QQWSSYPLT

SEQ ID NO: 251 - protein, 9 aa, Mus musculus
FQGSHVPYT

SEQ ID NO: 252 - protein, 9 aa, Mus musculus
QQSNKDPRT

SEQ ID NO: 253 - protein, 9 aa, Mus musculus
QNGHSFPLT

SEQ ID NO: 254 - protein, 9 aa, Mus musculus
QQSIEDPWT

SEQ ID NO: 255 - protein, 9 aa, Mus musculus
QQHYSTPYT

SEQ ID NO: 256 - protein, 9 aa, Mus musculus
QQYSSYPLT

SEQ ID NO: 257 - protein, 9 aa, Mus musculus
QQHYSTPPT

SEQ ID NO: 258 - protein, 10 aa, Mus musculus
GQSYSYPPFT

SEQ ID NO: 259 - protein, 9 aa, Mus musculus
QQGNTLPWT

SEQ ID NO: 260 - protein, 9 aa, Mus musculus
HQFSSYPLT

SEQ ID NO: 261 - protein, 9 aa, Mus musculus
QQSNKDPLT

SEQ ID NO: 262 - protein, 9 aa, Mus musculus
QHSRELPLT

SEQ ID NO: 263 - protein, 9 aa, Mus musculus
AQNLELPWT

SEQ ID NO: 264 - protein, 9 aa, Mus musculus
SQSTHVPPT

SEQ ID NO: 265 - protein, 9 aa, Mus musculus
WQGTHFPQT

SEQ ID NO: 266 - protein, 9 aa, Mus musculus
QQWSSNPYT

SEQ ID NO: 267 - protein, 9 aa, Mus musculus
QQYSSYPYT

SEQ ID NO: 268 - protein, 9 aa, Mus musculus
QQYSSYTWT

SEQ ID NO: 269 - protein, 9 aa, Mus musculus
LQYDEFPYT

SEQ ID NO: 270 - protein, 9 aa, Mus musculus
GQSYSYPFT

SEQ ID NO: 271 - protein, 9 aa, Mus musculus
QHHYVSPRT

SEQ ID NO: 272 - protein, 15 aa, synthetic construct
GGGGSGGGGSGGGGS

SEQ ID NO: 273 - protein, 105 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAY

SEQ ID NO: 274 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 275 - protein, 21 aa, Homo sapiens
IYIWAPLAGTCGVLLLSLVIT

SEQ ID NO: 276 - protein, 28 aa, Homo sapiens
IYIWAPLAGTCGVLLLSLVITLYCNHRN

SEQ ID NO: 277 - protein, 27 aa, Homo sapiens
FWVLVVVGGVLACYSLLVTVAFIIFWV

SEQ ID NO: 278 - protein, 42 aa, Homo sapiens
ALYLLRRDQRLPPDAHKPPGGGSFRTPIQEEQADAHSTLAKI

SEQ ID NO: 279 - protein, 42 aa, Homo sapiens
KRGRKKLLYIFKQPFMRPVQTTQEEDGCSCRFPEEEEGGCEL

SEQ ID NO: 280 - protein, 21 aa, Homo sapiens
GSGEGRGSLLTCGDVEENPGP

SEQ ID NO: 281 - protein, 22 aa, Homo sapiens
GSGATNFSLLKQAGDVEENPGP

SEQ ID NO: 282 - protein, 23 aa, Homo sapiens
GSGQCTNYALLKLAGDVESNPGP

SEQ ID NO: 283 - protein, 25 aa, Homo sapiens
GSGVKQTLNFDLLKLAGDVESNPGP

SEQ ID NO: 284 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 285 - protein, 260 aa, Homo sapiens
MAQAALEPGEKPYACPECGKSFSTSGSLVRHQRTHTGEKPYKCPECGKSFSRNDALTEHQRTHTGEKPYKCPECGKSFSSKKHLAEHQRTHTGEKPYACPECGKSFSTSGELVRHQRTHTGEKPYKCPECGKSFSRSDKLVRHQRTHTGEKPYKCPECGKSFSRSDHLTEHQRTHTGKKTSGQAGQASPKKKRKVGRADALDDFDLDMLGSDALDDFDLDMLGSDALDDFDLDMLGSDALDDFDLDMLINYPYDVPDYAS

SEQ ID NO: 286 - protein, 211 aa, Homo sapiens
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS